SEQUENCE LISTING (32 sequences)

SEQ ID NO: 1 - protein, 14 aa, Artificial Sequence
  Description of Artificial Sequence  Synthetic Peptide
KLAKLAKKLAKLAK

SEQ ID NO: 2 - protein, 14 aa, Artificial Sequence
  Description of Artificial Sequence  Synthetic Peptide
KLAKKLAKLAKKLA

SEQ ID NO: 3 - protein, 14 aa, Artificial Sequence
  Description of Artificial Sequence  Synthetic Peptide
KAAKKAAKAAKKAA

SEQ ID NO: 4 - protein, 21 aa, Artificial Sequence
  Description of Artificial Sequence  Synthetic Peptide
KLGKKLGKLGKKLGKLGKKLG

SEQ ID NO: 5 - protein, 7 aa, Artificial Sequence
  Description of Artificial Sequence  Synthetic Peptide
AYHRLRR

SEQ ID NO: 6 - protein, 7 aa, Artificial Sequence
  Description of Artificial Sequence  Synthetic Peptide
GFYWLRS

SEQ ID NO: 7 - protein, 7 aa, Artificial Sequence
  Description of Artificial Sequence  Synthetic Peptide
SFFYLRS

SEQ ID NO: 8 - protein, 15 aa, Artificial Sequence
  Description of Artificial Sequence  Synthetic Peptide
ADGACPCFLLGCCGA

SEQ ID NO: 9 - protein, 7 aa, Artificial Sequence
  Description of Artificial Sequence  Synthetic Peptide
LRSGAGS

SEQ ID NO: 10 - protein, 7 aa, Artificial Sequence
  Description of Artificial Sequence  Synthetic Peptide
ATGRVLG

SEQ ID NO: 11 - protein, 7 aa, Artificial Sequence
  Description of Artificial Sequence  Synthetic Peptide
ILGGGHA

SEQ ID NO: 12 - protein, 7 aa, Artificial Sequence
  Description of Artificial Sequence  Synthetic Peptide
DFSGIAX

SEQ ID NO: 13 - protein, 6 aa, Artificial Sequence
  Description of Artificial Sequence  Synthetic Peptide
GRSGXR

SEQ ID NO: 14 - protein, 6 aa, Artificial Sequence
  Description of Artificial Sequence  Synthetic Peptide
SGASAV

SEQ ID NO: 15 - protein, 6 aa, Artificial Sequence
  Description of Artificial Sequence  Synthetic Peptide
SGXGVF

SEQ ID NO: 16 - protein, 4 aa, Artificial Sequence
  Description of Artificial Sequence  Synthetic Peptide
AGSF

SEQ ID NO: 17 - protein, 6 aa, Artificial Sequence
  Description of Artificial Sequence  Synthetic Peptide
SLGSFP

SEQ ID NO: 18 - protein, 4 aa, Artificial Sequence
  Description of Artificial Sequence  Synthetic Peptide
LVSA

SEQ ID NO: 19 - protein, 4 aa, Artificial Sequence
  Description of Artificial Sequence  Synthetic Peptide
WSGL

SEQ ID NO: 20 - protein, 4 aa, Artificial Sequence
  Description of Artificial Sequence  Synthetic Peptide
GWSG

SEQ ID NO: 21 - protein, 6 aa, Artificial Sequence
  Description of Artificial Sequence  Synthetic Peptide
XSVLTR

SEQ ID NO: 22 - protein, 4 aa, Artificial Sequence
  Description of Artificial Sequence  Synthetic Peptide
SLGG

SEQ ID NO: 23 - protein, 4 aa, Artificial Sequence
  Description of Artificial Sequence  Synthetic Peptide
GSLS

SEQ ID NO: 24 - protein, 5 aa, Artificial Sequence
  Description of Artificial Sequence  Synthetic Peptide
LSLSL

SEQ ID NO: 25 - protein, 6 aa, Artificial Sequence
  Description of Artificial Sequence  Synthetic Peptide
XPGSSF

SEQ ID NO: 26 - protein, 6 aa, Artificial Sequence
  Description of Artificial Sequence  Synthetic Peptide
GSSXWA

SEQ ID NO: 27 - protein, 4 aa, Artificial Sequence
  Description of Artificial Sequence  Synthetic Peptide
PGLL

SEQ ID NO: 28 - protein, 5 aa, Artificial Sequence
  Description of Artificial Sequence  Synthetic Peptide
AGVGV

SEQ ID NO: 29 - protein, 6 aa, Artificial Sequence
  Description of Artificial Sequence  Synthetic Peptide
XCFGGX

SEQ ID NO: 30 - protein, 9 aa, Artificial Sequence
  Description of Artificial Sequence  Synthetic Peptide
CRVASVLPC

SEQ ID NO: 31 - protein, 9 aa, Artificial Sequence
  Description of Artificial Sequence  Synthetic Peptide
CYAIGSFDC

SEQ ID NO: 32 - protein, 4 aa, Artificial Sequence
  Description of Artificial Sequence  Synthetic Peptide
SWSG

(19) United States
(12) Patent Application Publication
(10) Pub. No.: US 2012/0270808 A1
(43) Pub. Date: Oct. 25, 2012

(54) COMPOSITIONS AND METHODS RELATED TO SYNCHRONOUS SELECTION OF HOMING PEPTIDES FOR MULTIPLE TISSUES BY IN VIVO PHAGE DISPLAY

(76) Inventors: Mikhail Kolonin, Houston, TX (US); Wadih Arap, Houston, TX (US); Renata Pasqualini, Houston, TX (US)

(21) Appl. No.: 13/439,172

(22) Filed: Apr. 4, 2012

Related U.S. Application Data
(63) Continuation of application No. 11/719,510, filed on May 5, 2009, now abandoned.
(60) Provisional application No. 60/628,495, filed on Nov. 16, 2004.

(51) Int. Cl. A61K 38/16; C12N 7/00; C40B 30/04; A61K 38/10; A61K 38/08; A61K 38/07; A61K 38/06; C07K 14/00; C07K 7/06; C07K 7/08; C07K 14/52; C07K 14/57; C07K 14/565; C07K 14/555; C07K 14/525; C07K 14/53; C07K 14/55; C07K 14/54; C07K 14/56; C07K 14/545; C12N 11/00

(52) U.S. Cl. 514/21.2; 435/174; 435/235.1; 506/9; 514/21.3; 514/21.4; 514/21.5; 514/21.6; 514/21.7; 514/21.8; 514/21.9; 530/324; 530/325; 530/326; 530/327; 530/328; 530/329; 530/330; 530/331; 530/350; 530/351; 530/387.3; 530/399

(57) ABSTRACT

Embodiments of the invention include methods for selecting in parallel (i.e., synchronously or simultaneously) peptides that target a number of organs, in which each peptide targets distinct tissues or organs. Typically, the methods of the invention provide for peptide selection in a Minimal number of subjects and still provides a selectively binding peptide. In certain aspects, methods of identifying peptides that bind to multiple selected tissues or organs of an organism may comprise the steps of administering a phage display library to a first subject; obtaining a sample of two or more selected tissues; obtaining phage displaying peptides that bind to the samples from the first subject; enriching for peptides by administering phage isolated from the samples of the first subject to a second subject; obtaining a sample of two or more selected tissues from the second subject; and identifying the peptides displayed.

This application claims priority to U.S. Provisional Patent application Ser. No. 60/628,495, filed Nov. 16, 2004, which is incorporated herein by reference in its entirety

The United States Government owns rights in this invention pursuant to grant numbers CA103030, DK67683, CA90810, and CA90270 from the National Institutes of Health, and grant number BC023663 from the Department of Defense Further support was provided by the Gillson-Longenbaugh Foundation

### BACKGROUND OF THE INVENTION

I. Field of the Invention

The present invention concerns the fields of molecular medicine and targeted delivery of therapeutic or diagnostic agents More specifically, the present invention relates to compositions and methods for identification and use of peptides that target various tissues of an organism

II. Description of Related Art

Vascular mapping by in vivo phage display reveals selectively expressed biochemical “addresses” within different vasculatures This type of approach has uncovered ligand-receptor systems that can be used for the delivery of agents to specific tissues (Arap et al, 1998, Pasqualini et al, 1996, Arap et al, 2002, Kolonin et al, 2001, Pasqualini et al, 2000) The screening is based on the preferential ability of short ligand peptides from combinatorial libraries (displayed on the pIII protein of an M 13-based phage vector) to home to a specific organ after systemic administration (Pasqualini et al, 2000) Peptides targeting tissues and disease states have been isolated and, in some cases, led to the identification of the corresponding vascular receptors (Arap et al, 1998, Pasqualini et al, 1996, Arap et al, 2002, Kolonin et al, 2001, Rajotte and Ruoslahti, 1999, Kolonin, et al, 2002, Kolonin et al, 2004) Recently, the inventors have reported the screening of a phage display library in a cancer patient, one of the ligand motifs has been identified as an interleukin-11-like peptide and its homing to the interleukin-11 receptor is being exploited as a potential strategy for targeted therapeutic delivery in human prostate cancer (Zurita et al, 2004)

So far, a rate-limiting step of the selection of phage display random peptide libraries in vivo has been the requirement of three to four rounds of selection in order to enrich for the best homing motifs (Pasqualini et al, 2000) While it is possible to obtain ligand peptides after single round of screening (Arap et al, 2002, Zurita et al, 2004) by greatly increasing the number of peptides recovered and surveyed, there are considerable practical limitations to the number of phage clones that can be processed Such limitations are particularly important in the context of screening in patients since maximal information recovery is critical, to meet this challenge additional protocols for efficient discovery of homing ligands to human biological addresses need to be developed

### SUMMARY OF THE INVENTION

Embodiments of the invention include methods for selecting in parallel (i e, synchronously or simultaneously) peptides that target a number of organs, in which each peptide targets distinct tissues or organs Typically, the methods of the invention provide for peptide selection in a minimal number of subjects and provide selectively binding peptides independently for individual organs In certain aspects, methods of identifying peptides that bind to multiple selected tissues or organs of an organism may comprise the steps of a) administering a phage display library to a first subject, b) obtaining a sample of two or more selected tissues from the first subject, c) obtaining phage displaying peptides that bind to the samples from the first subject, d) enriching for peptides corresponding to the phage obtained in step c that bind a selected tissue by administering phage corresponding to the phage isolated from the samples of the first subject to a second subject, e) obtaining a sample of two or more selected tissues from the second subject, and f) identifying the peptides displayed by the phage isolated from the samples of the second subject. The procedure described for a-c can be repeated for any desired number of total selection rounds (typically 3-4) The term “phage display library” refers to a plurality of phage in which a random heterologous peptide has been engineered into a phage coat protein and presented on the phage surface In certain aspects, the peptide may be constrained by cysteine residues of the peptide The methods may further comprise administering phage isolated from the second subject to at least a third subject, obtaining samples of one or more tissues from the third subject, and identifying the peptide sequence displayed by phage isolated from the tissues of the third subject In certain aspects, the administration of phage is by injection, preferably intravenous injection The subject may be a mammal, and in particular aspects the mammal is a human

The methods may further comprise amplifying the phage isolated from the samples of one subject prior to administration to an additional subject Amplifying may entail PCR amplification of all or part of a phage nucleic acid followed by cloning the ampified fragment into a second phage, and/or multiplication of phage through a phage host organism, e g, bactena that support phage replication In certain aspects, phage are recovered by Biopanning and Rapid Analysis of Selective Interactive Ligands (BRASIL) Samples may be derived from various organs in parallel, that is by obtaining samples from a subject at about the same time The term simultaneously or synchronously may be used to mean that samples are obtained in a time interval (thirty minutes to hours) that accommodates the taking of samples from multiple sites in a subject An organ may include, but is not limited to, muscle, pancreas, brain, kidney, uterus, bowel, intestine, small intestine, heart, artery, vein, aorta, coronary artery, lung, spleen, bone marrow, bladder, prostate, adipose, ovary or any other tissue or organ known to one of skill in the art The methods may further comprise obtaining a sample from one or more non-selected tissue or organ, exposing the sample to the phage display library, recovenng the phage that are not bound to the non-selected tissue or organ, and subjecting the recovered phage to the methods described herein

Other embodiments of the invention include isolated peptides identified by the methods described herein In certain aspects, an isolated peptide is 100 amino acids or less in size, comprising at least 3 contiguous amino acids of a sequence selected from the group consisting of Ala-Pro-Ala (APA), Arg-Ser-Gly (RSG), Ser-Gly-Ala (SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GWS), Ile-Ala-Tyr (IAY), Arg-Arg-Ser (RRS), Ser-Gly-Val (SGV), Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), Phe-Gly-Val (FGV), Leu-Gly-Gly (LGG), Arg-Gly-Phe (RGF), Ala-Leu-Gly (ALG), Leu-Leu-Ser (LLS), Asp-Ser-Tyr (DSY), Gly-Phe-Ser (GFS), Gly-Ile-Trp (GIW), His-Gly-Leu (HGL), Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), Phe-Leu-Ser (FLS), Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), Gly-Pro-Ser (GPS), Ala-Gly-Val (AGV), Trp-Arg-Asp (WRD), Phe-Gly-Gly (FGG), Gly-Gly-Arg (GGR), Gly-Arg-Val (GRV), Arg-Trp-Ser (RWS), Val-Gly-Val (VGV), and Gly-Val-Gly (GVG), wherein the peptide selectively binds a tissue or organ In other aspects the isolated peptide may be 3, 4, 5, 6, 7, 8, 9, 10, 12, 14, 16, 18, 20, 22, 24, 25, 30, 35, 40, 45 or 50 amino acids in size, including lengths therebetween In particular aspects, the peptides are cyclic peptides

In still further aspects, the isolated peptide may comprise an amino acid sequence selected from the group consisting of Asp-Phe-Ser-Gly-Ile-Ala-Xaa (SEQ ID NO 12), Gly-Arg-Ser-Gly-Xaa-Arg (SEQ ID NO 13), Ser-Gly-Ala-Ser-Ala-Val (SEQ ID NO 14), Ser-Gly-Xaa-Gly-Val-Phe (SEQ ID NO 15), Ala-Gly-Ser-Phe (SEQ ID NO 16), Ser-Leu-Gly-Ser-Phe-Pro (SEQ ID NO 17), Leu-Val-Ser-Ala (SEQ ID NO 18), Trp-Ser-Gly-Leu (SEQ ID NO 19), Gly-Trp-Ser-Gly (SEQ ID NO 20), Xaa-Ser-Val-Leu-Thr-Arg (SEQ ID NO 21), Ser-Leu-Gly-Gly (SEQ ID NO 22), Gly-Ser-Leu-Ser (SEQ ID NO 23), Leu-Ser-Leu-Ser-Leu (SEQ ID NO 24), Xaa-Pro-Gly-Ser-Ser-Phe (SEQ ID NO 25), Gly-Ser-Ser-Xaa-Trp-Ala (SEQ ID NO 26), Pro-Gly-Leu-Leu (SEQ ID NO 27), Ala-Gly-Val-Gly-Val (SEQ ID NO 28), and Xaa-Cys-Phe-Gly-Gly-Xaa (SEQ ID NO 29), wherein Xaa is a positively charged amino acid

Isolated peptides of the invention may be operatively coupled to an agent to be delivered to a tissue, organ, or vasculature thereof Aspects of the invention include peptides that are covalently coupled to the agent to be delivered The agent may be a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, an imaging agent, survival factor, an anti-apoptotic agent, a hormone antagonist or an antigen

In a further aspect of the invention, a pro-apoptosis agent may be selected from the group consisting of gramicidin, magamin, mellitin, defensm, cecropin, (KLAKLAK)₂ (SEQ ID NO 1), (KLAKKLA)₂ (SEQ ID NO 2), (KAAKKAA)₂ (SEQ ID NO 3) and/or (KLGKKLG)₃ (SEQ ID NO 4) In a still futher aspect, an anti-angiogenic agent may be selected from the group consisting of thrombospondin, angiostatin 5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferm-related protein, carboxiamidotriazole, CM101 Marimastat, pentosan polysulphate, angiopoietm 2 (Regeneron), interferon-alpha, herbimycin A, PNU145156E, 16K prolactin fragment, Linomide, thalidomide, pentoxifylline, genistein, TNP-470, endostatin, pachtaxel, Docetaxel, polyamines, a proteasome inhibitor, a kinase inhibitor, a signaling peptide, accutin, cidofovir, vincristme, bleomycin, AGM-1470, platelet factor 4 and minocycline In yet another aspect, a cytokine may be selected from the group consisting of interleukin 1 (IL-1), IL-2, IL-5, IL-10, IL-11, IL-12, IL-18, interferon-γ (IF-γ), IF-α, IF-β, tumor necrosis factor-α (TNF-α), or GM-CSF (granulocyte macrophage colony stimulating factor)

In still further embodiments of the invention, the agent may be a virus, a bacteriophage, a bacterium, a liposome, a microparticle, a magnetic bead, a yeast cell, a mammalian cell or a cell In certain aspects, the virus is a lentivirus, a papovaviruses, a simian virus 40, a bovine papilloma virus, a polyoma virus, adenovirus, vaccinia virus, adeno-associated virus (AAV), or herpes virus The agent may also be a eukaryotic expression vector, and more preferably a gene therapy vector The isolated peptides of the invention may be attached to a solid support, e g, an array or bead

In yet still further embodiments of the invention a peptide may be a muscle-targeting peptide compnsing a three amino acid sequence selected from the group consisting of Ala-Pro-Ala (APA), Arg-Ser-Gly (RSG), Ser-Gly-Ala (SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GWS), Ile-Ala-Tyr (IAY), Arg-Arg-Ser (RRS), and Ser-Gly-Val (SGV) In certain aspects, the muscle-targeting peptide comprises an amino acid sequence selected from the group consisting of Asp-Phe-Ser-Gly-Ile-Ala-Xaa (SEQ ID NO 12), Gly-Arg-Ser-Gly-Xaa-Arg (SEQ ID NO 13), Ser-Gly-Ala-Ser-Ala-Val (SEQ ID NO 14), Ser-Gly-Xaa-Gly-Val-Phe (SEQ ID NO 15), Ala-Gly-Ser-Phe (SEQ ID NO 16), and Ser-Leu-Gly-Ser-Phe-Pro (SEQ ID NO 17), wherein Xaa is a positively charged amino acid

Embodiments of the invention include an isolated pancreas-targeting peptide comprising a three amino acid sequence selected from the group consisting of Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), and Phe-Gly-Val (FGV), wherein Xaa is a positively charged amino acid In certain aspects, the isolated peptide compnses an amino acid sequence selected from the group consisting of Leu-Val-Ser-Ala (SEQ ID NO 18), Trp-Ser-Gly-Leu (SEQ ID NO 19), Gly-Trp-Ser-Gly (SEQ ID NO 20), and Xaa-Ser-Val-Leu-Thr-Arg (SEQ ID NO 21), wherein Xaa is a positively charged amino acid

Still further embodiments of the invention include an isolated brain-targeting peptide comprising a three amino acid sequence selected from the group consisting of Leu-Gly-Gly (LGG), Arg-Gly-Phe (RGF), Ala-Leu-Gly (ALG), Leu-Leu-Ser (LLS), Asp-Ser-Tyr (DSY), Gly-Phe-Ser (GFS), Gly-Ile-Trp (GIW), and His-Gly-Leu (HGL) In certain aspects, the brain-targeting peptide comprises an amino acid sequence of Ser-Leu-Gly-Gly (SEQ ID NO 22)

In yet further embodiments of the invention, an isolated kidney-targeting peptide may comprise a three amino acid sequence selected from the group consisting of Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), and Phe-Leu-Ser (FLS) In certain aspects, the isolated peptide comprises an amino acid sequence of Gly-Ser-Leu-Ser (SEQ ID NO 23) or Leu-Ser-Leu-Ser-Leu (SEQ ID NO 24)

Embodiments also include an isolated uterus-targeting peptide, comprising a three amino acid sequence selected from the group consisting of Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), and Gly-Pro-Ser (GPS) In certain aspects the uterus-targeting peptide comprises an amino acid sequence selected from the group consisting of Xaa-Pro-Gly-Ser-Ser-Phe (SEQ ID NO 25), Gly-Ser-Ser-Xaa-Trp-Ala (SEQ ID NO 26), and Pro-Gly-Leu-Leu (SEQ ID NO 27), wherein Xaa is a positively charged amino acid

In further embodiments of the invention, an isolated bowel-targeting peptide may comprise a three amino acid sequence selected from the group consisting of Ala-Gly-Val (AGV), Trp-Arg-Asp (WRD), Phe-Gly-Gly (FGG), Gly-Gly-Arg (GGR), Gly-Arg-Val (GRV), Arg-Trp-Ser (RWS), Val-Gly-Val (VGV), and Gly-Val-Gly (GVG) Aspects of the invention include a bowel-targeting peptide comprising an amino acid sequence of Ala-Gly-Val-Gly-Val (SEQ ID NO 28), or Xaa-Cys-Phe-Gly-Gly-Xaa (SEQ ID NO 29), wherein Xaa is a positively charged amino acid

Embodiments of the invention may also include an isolated peptidomimetic comprising a sequence that mimics a peptide selected from the group consisting of Ala-Pro-Ala (APA), Arg-Ser-Gly (RSG), Ser-Gly-Ala (SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GWS), Ile-Ala-Tyr (IAY), Arg-Arg-Ser (RRS), Ser-Gly-Val (SGV), Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), Phe-Gly-Val (FGV), Leu-Gly-Gly (LGG), Arg-Gly-Phe (RGF), Ala-Leu-Gly (ALG), Leu-Leu-Ser (LLS), Asp-Ser-Tyr (DSY), Gly-Phe-Ser (GFS), Gly-Ile-Trp (GIW), His-Gly-Leu (HGL), Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), Phe-Leu-Ser (FLS), Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), Gly-Pro-Ser (GPS), Ala-Gly-Val (AGV), Trp-Arg-Asp (WRD), Phe-Gly-Gly (FGG), Gly-Gly-Arg (GGR), Gly-Arg-Val (GRV), Arg-Trp-Ser (RWS), Val-Gly-Val (VGV), and Gly-Val-Gly (GVG), wherein the sequence selectively binds to a tissue or organ

Further embodiments include methods of targeting the delivery of an agent to a tissue, organ, or vasculature thereof, in a subject, by obtaining an inventive peptide as described herein or according to the inventive methods described herein, operatively coupling the peptide to the agent, and administering the peptide-coupled agent to the subject A subject may be, but is not limited to, a primate, a monkey, a human, a mouse, a dog, a cat, a rat, a sheep, a horse, a cow, a goat or a pig The agent can be a drug, a chemotherapeutic agent, a radioisotope, a pro-apoptosis agent, an anti-angiogenic agent, an enzyme, a hormone, a cytokine, a growth factor, a cytotoxic agent, a peptide, a protein, an antibiotic, an antibody, a Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdcvice, a yeast cell, a mammalian cell, a cell or an expression vector

In yet further embodiments, methods of identifying a receptor or protein that interacts with a tissue or organ selective peptide comprise the steps of obtaining a composition suspected of comprising a receptor or protein that interacts with a tissue or organ selective peptide, contacting the composition with a peptide of the invention or identified by the methods of the invention under conditions that permit binding of the peptide to any such receptor or protein present in the composition, and identifying a receptor or protein that binds to the peptide The methods may include the step of isolating the receptor or protein, preparing an antibody or antibody fragment that recognizes and binds to the receptor or protein, or the like An agent that one desires to have delivered to the tissue or organ may be attached to the antibody or antibody fragment

Embodiments of the invention also include an antibody or antibody fragment that recognizes and binds to a receptor or protein identified by the methods of the invention The antibody or antibody fragment may further comprise an agent or macromolecular complex that one desires to have delivered to a selected tissue, organ, or vascular target attached to the antibody or antibody fragment

Certain embodiments concern methods of obtaining antibodies against an antigen In preferred embodiments, the antigen comprises one or more targeting peptides The targeting peptides are prepared and immobilized on a solid support, serum containing antibodies is added and antibodies that bind to the targeting peptides are collected

It is contemplated that any method or composition described herein can be implemented with respect to any other method or composition described herein

As used herein in the specification, “a” or “an” may mean one or more As used herein in the claim(s), in conjunction with the word “comprising,” the words “a” or “an” may mean one or more than one As used herein “another” may mean at least a second or more of an item

The use of the term “or” in the claims is used to mean “and/or” unless explicitly indicated to refer to alternatives only or the alternatives are mutually exclusive, although the disclosure supports a definition that refers to only alternatives and “and/or ”

Throughout this application, the term “about” is used to indicate that a value includes the standard deviation of error for the device or method being employed to determine the value

Other objects, features and advantages of the present invention will become apparent from the following detailed description It should be understood, however, that the detailed description and the specific examples, while indicating specific embodiments of the invention, are given by way of illustration only, since various changes and modifications within the spirit and scope of the invention will become apparent to those skilled in the art from this detailed description

### BRIEF DESCRIPTION OF THE DRAWINGS

The following drawings form part of the present specification and are included to further demonstrate certain aspects of the present invention The invention may be better understood by reference to one or more of these drawings in combination with the detailed description of specific embodiments presented herein

FIG. 1. A schematic description of synchronous in vivo phage display screening In every selection round, phage are intravenously administered and simultaneously recovered from “n” target tissues, amplified, pooled, and used for the next selection round Increased recovery of phage transforming units (TU) in every subsequent round reflects the selection of peptides preferentially homing to the target organ

FIG. 2. Monte Carlo simulations to assess tripeptide motif tissue homing For each selection round, all tripeptides isolated from the target organs were pooled with tripeptides isolated from the unselected CX₇C library Fisher's exact test was then performed on 1,000 random permutations of the experiment dataset For every permutation, the pool of tripeptides was randomly distributed into groups corresponding to numbers of peptide sequences used for the analysis (Table 1B) Plotted are the 50 smallest P-values (index number of P-values 1 through 50, ascending order) generated in each of the 1,000 permutations, as compared with the 50 smallest P-values determined in the actual data analysis, as described (Table 1B)

FIG. 3. Identification of extended motifs homing to mouse tissues Peptide sequences containing tripeptides enriched in a given tissue (Table 1) were aligned in clusters with ClustalW software to obtain longer motifs shared between different peptides from each cluster Similarity between peptides at the level of amino acid class is coded hydrophobic, neutral and polar, basic, or acidic Original tripeptides are depicted in bold, extended motifs are highlighted

FIGS. 4A-4B. Retro-BLAST analysis to identify PRLR ligand-matching motifs (FIG. 4A) Peptide sequences isolated from the pancreas-homing phage pool as those binding to PRLR were matched in each orientation to mature sheep (oPL) and mouse (mPL-I and mPRL) protein sequences (leader peptide sequence not included) Peptide matches of four or more residues in any position being identical to the corresponding amino acid positions in any of the three PRL homologues are displayed Shaded protein sequences published PRLR binding sites Motifs SGATGRA, SGPTGRA, QVHSSAY, VFSDYKR, and LPTLSLN were isolated by biopanning on both in vitro immobilized and cell-surface expressed PRLR Forward and reverse matches of the validated RVASVLP motif are underlined (FIG. 4B) Binding of pancreas-homing phagepeptides (recovered from synchronous biopanning rounds 2 and 3) to recombinant rabbit PRLR, as compared to their binding to BSA control TU transforming units

FIGS. 5A-5H. Validation of PRLR as a candidate receptor for a PRLR ligand mimic CRVASVLPC (FIG. 5A) Specific binding of the CRVASVLPC-phage, but not of the control phage (CYAIGSFDC-displaying or insertless fd-tet) to COS-1 cells transfected with pECE-PRLR Phage binding to COS-1 PRLR-transfected cells (as compared to control non-transfected cells) was determined by BRASIL (FIG. 5B) Binding of phage displaying forward SVL-containing CRVASVLPC motif (right arrow), as well as the reverse CPLVSAVRC motif (left arrow), to PRLR-transfected COS-1 cells, as compared to biding of the six alanine-scan motif mutants (A1 CAVASVLPC, A2 CRAASVLPC, A3 CRVAAVLPC, A4 CRVASALPC, A5 CRVASVAPC, and A6 CRVASVLAC) (FIGS. 5C-5D) Specific binding to and internalization of CRVASVLPC-phage (FIG. 5C) and an alanine-scan mutant A4 (FIG. 5D) into COS-1 PRLR-transfected cells, detected by co-immunolocalization of CRVASVLPC-phage with PRLR-expression, resulting in overlapping signal (FIG. 5C) (FIG. 5E-5H) Anti-phage immunohistochemistry in paraffin sections of formalin-fixed pancreas (FIGS. 5E and 5H) or skeletal muscle (FIG. 5F and 5G) from mice intravenously injected with CRVASVLPC-phage (FIGS. 5E and 5G), or control muscle-homing CYAIGSFDC-phage (FIGS. 5F and 5H)

### DESCRIPTION OF ILLUSTRATIVE EMBODIMENTS

Additional methods for identification of multiple peptides that selectively bind to tissues, organs or the vasculature thereof are still needed Embodiments of the invention include comprehensive integrated methods to synchronously or simultaneously identify homing ligands for multiple tissues in a screen In one aspect of the invention, the inventors have employed Biopanning and Rapid Analysis of Selective Interactive Ligands (BRASIL) to identify, in parallel, peptides that selectively bind to a variety of tissues, organs, and/or vasculature thereof As used herein “selective binding” in no way precludes binding to other cells or material, but connotes the preferential binding of a target tissue, organ, or vasculature thereof Selective binding may include a 2, 3, 4, 5, 6, 7, 8, 9, 10 or more fold preference for a selected tissue as compared to a non-selected tissue In one example, a plurality of tissues were profiled at the same time, i e, synchronously or simultaneously Screening of selected tissues with a CX₇C random phage library, for example, yielded several peptide motifs that selectively bound different tissues as compared to insertless phage or other negative controls Comparison of the selected motifs with available sequences in on-line protein databases suggests that a number of candidate proteins share homologous sequences with these peptides These peptides are being use in further studies to identify and purify protein(s) that interact, directly or indirectly, with an identified peptide, including identifying and purifying corresponding receptor(s) In the clinics the newly identified peptides and peptide motifs may serve as targeting moieties, drugs and/or drug leads Also, the identified peptides can be optimized as delivery vehicles or enhancers for targeted therapy of a selected tissue, organ, or vasculature thereof Methods of the present invention provide for the synchronous selection of homing peptides for multiple tissues and also provide additional methods for screening combinatorial libraries in vivo This approach adds new possibilities for efficient and quick identification of ligand-receptor pairs for therapeutic targeting In particular, the high-throughput screening afforded by these methods are well suited for mapping of human vascular addresses

A “targeting peptide” as used herein is a peptide comprising a contiguous sequence of amino acids, which is characterized by selective localization to an organ, tissue or cell type Selective localization may be determined, for example, by methods disclosed below, wherein the putative targeting peptide sequence is incorporated into a protein that is displayed on the outer surface of a phage Administration to a subject of a library of such phage that have been genetically engineered to express a multitude of such targeting peptides of different amino acid sequence is followed by collection of a plurality of tissues or organs derived from one or more subjects and identification of phage found in or associated with that tissue or organ A phage expressing a targeting peptide sequence is considered to be selectively localized to a tissue or organ if it exhibits greater binding or localization in that tissue or organ as compared to a control tissue or organ Preferably, selective localization of a targeting peptide should result in a two-fold or higher enrichment of the phage or peptide in the target tissue or organ, compared to a control tissue or organ Selective localization resulting in at least a three-fold, four-fold, five-fold, six-fold, seven-fold, eight-fold, nine-fold, ten-fold or higher enrichment in the target tissue or organ, as compared to a control organ, is more preferred

Alternatively, a phage expressing a targeting peptide sequence that exhibits selective localization preferably shows an increased enrichment in the target tissue or organ as compared to a control tissue or organ when phage recovered from the target or selected tissue or organ are injected into or put in contact with a second, third, fourth or more subjects for additional screening

Another alternative means to determine selective localization or binding is that phage expressing the putative target peptide preferably exhibit a two-fold, more preferably a three-fold or higher ennehment in the target tissue or organ as compared to control phage that express a non-specific peptide or that have not been genetically engineered to express any putative target peptides Yet another means to determine selective localization is that localization to the target organ of phage expressing the target peptide is at least partially blocked by the co-administration of a synthetic peptide containing the target peptide sequence “Targeting peptide” and “homing peptide” are used synonymously herein

### I. Synchronous Phage Library Screening on Multiple-Organs

In certain instances one may desire or is restricted to a limited number of subjects for peptide selection procedures In the* situations typical screening procedures are not optimal, thus the procedures described herein provide a more efficient method of identifying targeting peptides with characteristics amenable to development into drugs, targeting, or diagnositc agents In addition, the current methods used for phage display biopanning in the mouse model system require substantial improvement for use with humans Thus, improvements in the mouse system may be used to improve techniques utilized in humans Techniques for biopanning in human subjects are disclosed in PCT Patent Application PCT/US01/28044, filed Sep. 7, 2001 and in Arap et al, 2002, the entire text of which are incorporated herein by reference The methodology described herein is used to further enrich the selected phage population and to select various peptides targeting various organs in parallel or simultaneously A single screen in a single live patient selects a subpopulation of peptides, but this population needs to be enriched for selective peptides The inventor provides an improved methodolgy to acquire an enrichment of targeting peptides that may be utilized in, for example, human subjects

A “subject” refers generally to a mammal In certain preferred embodiments, the subject is a primate, a monkey, or a human In more preferred embodiments, the subject is a human In general, humans suitable for use with phage display are either brain dead or terminal wean patients The amount of phage library (preferably pnmary library) required for administration must be significantly increased, preferably to 10¹⁴ TU or higher, preferably administered intravenously in approximately 200 ml of Ringer lactate solution over about a 10 minute period

The amount of phage required for use in humans has required substantial improvement over the mouse protocol, increasing the amount of phage available for injection by five orders of magnitude To produce such large phage libraries, the transformed bactenal pellets recovered from up to 500 to 1000 transformations are amplified up to 10 times in the bacterial host, recovering the phage from each round of amplification and adding LB Tet medium to the bacterial pellet for collection of additional phage The phage inserts remain stable under these conditions and phage may be pooled to form the large phage display library required for humans Samples of vanous organs and tissues are collected starting approximately 15 minutes after injection of the phage library Samples are processed as described below and phage collected from each tissue or organ of interest for DNA sequencing to determine the amino acid sequences of targeting peptides

With humans, the opportunities for enrichment by multiple rounds of biopanning are severely restricted, compared to the mouse model system A substantial improvement in the biopanning technique involves polyorgan targeting wherein a variety of organs are targeted concurrently In the standard protocol for phage display biopanning, phage from a single organ are collected, amplified and injected into a new host, where tissue from the same organ is collected for phage rescue and a new round of biopanning However, the limited availability and expense of processing samples from humans requires improvements in the protocol

It is possible to pool phage collected from multiple organs after a first round of biopanning and inject the pooled sample into a new subject, where each of the multiple organs may be collected again for phage rescue The polyorgan targeting protocol may be repeated for as many rounds of biopanning as desired In this manner, it is possible to significantly reduce the number of subjects required for isolation of targeting peptides for multiple organs, while still achieving substantial enrichment of the tissue- or organ-homing phage

In preferred embodiments, phage are recovered from human tissues or organs after injection of a phage display library into a human subject In certain embodiments, phage may be recovered by exposing a sample of the tissue or organ to a pilus positive bacterium, such as E coli K91 In alternative embodiments, phage may be recovered by amplifying the phage inserts, ligating the inserts to phage DNA and producing new phage from the ligated DNA

### II. Identification of Targetting Peptides

The invention comprises methods for the identification of one or more targeting peptides or molecular targets that could be utilized for the localization of a composition to a particular tissue, organ or associated vasculature Screening of the tissues and organs of a subject with CXₙC, wherein n can be 4, 5, 6, 7, or more residues, random phage library that yield several peptide motifs In one example, various clones (comprising tripeptide motifs of Ala-Pro-Ala (APA), Arg-Ser-Gly (RSG), Ser-Gly-Ala (SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GWS), Ile-Ala-Tyr (IAY), Arg-Arg-Ser (RRS), Ser-Gly-Val (SGV), Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), Phe-Gly-Val (FGV), Leu-Gly-Gly (LGG), Arg-Gly-Phe (RGF), Ala-Leu-Gly (ALG), Leu-Leu-Ser (LLS), Asp-Ser-Tyr (DSY), Gly-Phe-Ser (GFS), Gly-Ile-Trp (GIW), His-Gly-Leu (HGL), Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), Phe-Leu-Ser (FLS), Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), Gly-Pro-Ser (GPS), Ala-Gly-Val (AGV), Trp-Arg-Asp (WRD), Phe-Gly-Gly (FGG), Gly-Gly-Arg (GGR), Gly-Arg-Val (GRV), Arg-Trp-Ser (RWS), Val-Gly-Val (VGV), or Gly-Val-Gly (GVG)) exhibited high frequency, selective binding to various tissues or organs Comparison of the selected motifs with available sequences in on-line protein databases suggests that a number of candidate proteins share homologous or similar sequences with these peptides Mechanistic studies surrounding these targets are being pursued to provide a rich platform for the identification of peptides for the targeting of various tissues, organs, and associated vasculature as well as combinations of such The findings will also have important clinical implications in that newly identified motifs may serve as a peptidomimetic drug leads and can he optimized to direct delivery of various therapeutic moities

Peptides of the invention may include various 3, 4, 5, 6, 7, 8, or more peptide motifs or amino acid sequences These motifs may include those that selectively bind one or more tissues or organs For example, a muscle-selective peptide may comprise Ala-Pro-Ala (APA), Arg-Ser-Gly(RSG), Ser-Gly-Ala(SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GTS), Ile-Ala-Tyr (IAY), Arg-Arg-Ser (RRS), and Ser-Gly-Val (SGV) peptide motifs Pancreas-selective peptide motifs include Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), and Phe-Gly-Val (FGV) Brain selective peptide motifs include Leu-Gly-Gly (LGG), Arg-Gly-Phe (RGF), Ala-Leu-Gly (ALG), Leu-Leu-Ser (LLS), Asp-Ser-Tyr (DST), Gly-Phe-Ser (GFS), Gly-Ile-Trp (GIW), and His-Gly-Leu (HGL) Kidney-selective peptides include Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), and Phe-Leu-Ser (FLS) Uterus-selective peptides include Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), and Gly-Pro-Ser (GPS) Bowel-selective peptide motifs include Ala-Gly-Val (AGV), Trp-Arg-Asp (WRN), Phe-Gly-Gly (FGG), Gly-Gly-Arg (GGR), Gly-Arg-Val (GRV), Arg-Trp-Ser (RWS), Val-Gly-Val (VGV), and Gly-Val-Gly (GVG)

BRASIL has been successfully used to isolate phage in various cell systems such as activated endothelial cells and tumor cells BRASIL has also been used to isolate bone marrow homing phage using in vivo/ex-vivo based strategies One method includes injecting the phage libraries intravenously and recover samples after a few minutes

A. Phage Display

Recently, an en vivo selection system was developed using phage display libranes to identify organ, tissue or cell type-targeting peptides in a mouse model system Phage display libraries expressing transgenic peptides on the surface of bacteriophage were initially developed to map epitope binding sites of immunoglobulins (Smith and Scott, 1985 and 1993) Such libraries can be generated by inserting random oligonucleotides into cDNAs encoding a phage surface protein, generating collections of phage particles displaying unique peptides in as many as 10⁹ permutations (Pasqualini and Ruoslahti, 1996, Arap et al, 1998a and 1998b)

A “phage display library” is a collection of phage that have been genetically engineered to express a set of putative targeting peptides on their outer surface In preferred embodiments, DNA sequences encoding the putative targeting peptides are inserted in frame into a gene encoding a phage capsule protein In other preferred embodiments, the putative targeting peptide sequences are in part random mixtures of all twenty amino acids and in part non-random In certain preferred embodiments the putative targeting peptides of the phage display library exhibit one or more cysteine residues at fixed locations within the targeting peptide sequence Cysteines may be used, for example, to create a cyclic peptide

Targeting peptides selective for a given organ, tissue or cell type can be isolated by “biopanning” (Pasqualini and Ruoslahti, 1996, Pasqualmi, 1999) In brief, a library of phage containing putative targeting peptides is administered to an animal or human, and samples of organs, tissues or cell types containing phage are collected In preferred embodiments utilizing filamentous phage, the phage may be propagated in vitro between rounds of biopanning in pilus-positive bacteria The bacteria are not lysed by the phage but rather secrete multiple copies of phage that display a particular insert Phage that bind to a target molecule can be eluted from the target organ, tissue or cell type and then amplified by growing them in host bacteria If desired, the ample fled phage can be administered to a host and samples of organs, tissues or cell types again collected Multiple rounds of biopanning can be performed until a population of selective binders is obtained The amino acid sequence of the peptides is determined by sequencing the DNA corresponding to the targeting peptide insert in the phage genome The identified targeting peptide can then be produced as a synthetic peptide by standard protein chemistry techniques (Arap et al, 1998a, Smith and Scott, 1985) This approach allows circulating targeting peptides to be detected in an unbiased functional assay, without any preconceived notions about the nature of their target Once a candidate target is identified as the receptor of a targeting peptide, it can be isolated, purified and cloned by using standard biochemical methods (Pasqualini, 1999, Rajotte and Ruoslahti, 1999)

In certain embodiments, a subtraction protocol may be used to further reduce background phage binding The purpose of subtraction is to remove phage from the library that bind to tissues other than the tissue of interest In alternative embodiments, the phage library may be prescreened against a subject who does not possess the selected tissues or organs For example, placenta-binding peptides may be identified after prescreening a library against a male or non-pregnant female subject After subtraction the library may be screened against the tissue or organ of interest Other subtraction protocols are known and may be used in the practice of the present invention, for examples see U.S. Pat. Nos. 5,840,841, 5,705,610, 5,670,312 and 5,492,807, which are incorporated herein by reference in their entirety

B. Biopanning and Rapid Analysis of Selective Interactive Ligands (BRASIL)

In preferred embodiments, separation of phage bound to the cells of a target organ, tissue or cell type from unbound phage is achieved using the BRASIL (Biopanning and Rapid Analysis of Soluble Interactive Ligands) technique (PCT Application PCT/US01/28124 entitled, “Biopanning and Rapid Analysis of Selective Interactive Ligands (BRASIL)” by Arap at al, filed Sep. 7, 2001, incorporated herein by reference in its entirety) In BRASIL, an organ sample, tissue sample or cell type is gently separated into cells or small clumps of cells that are suspended in an aqueous phase The aqueous phase is layered over an organic phase of appropriate density and centrifuged. Cells attached to bound phage are pelleted at the bottom of the centrifuge tube, while unbound phage remain in the aqueous phase This allows a more efficient separation of bound from unbound phage, while maintaining the binding interaction between phage and cell BRASIL may be performed in an in vivo protocol, in which organs, tissues or cell types are exposed to a phage display library by intravenous administration, or by an ex vivo protocol, where the cells are exposed to the phage library in the aqueous phase before centrifugation

C. Preparation of Large Scale Primary Libraries

In certain embodiments, primary phage libraries are amplified before injection into a subject A phage library is prepared by ligating targeting peptide-encoding sequences into a phage vector, such as fUSE5 The vector is transformed into pilus negative host E coli such as strain MC1061 The bacteria are grown overnight and then aliquots are frozen to provide stock for library production Use of pilus negative bacteria avoids the bias in libraries that arises from differential infection of pilus positive bactena by different targeting peptide sequences

To freeze, bacteria are pelleted from two thirds of a primary library culture (5 liters) at 4000×g for 10 mm Bacteria are resuspended and washed twice with 500 ml of 10% glycerol in water, then frozen in an ethanol/dry ice bath and stored at −80° C.

For amplification, 15 ml of frozen bacteria are inoculated into 5 liters of LB medium with 20 μg/ml tetracycline and grown overnight Thirty minutes after inoculation, a serial dilution is plated on LB/tet plates to verify the viability of the culture If the number of viable bacteria is less than 5-10 times the number of individual clones in the library (1-2×10⁸) the culture is discarded

After growing the bacterial culture overnight, phage are precipitated About ¼ to ⅓ of the bacterial culture is kept growing overnight in 5 liters of fresh medium and the cycle is repeated up to 5 times Phage are pooled from all cycles and used for injection into human subjects

Attachment of therapeutic agents to targeting peptides resulted in the selective delivery of the agent to a desired organ, tissue or cell type in the mouse model system Targeted delivery of chemotherapeutic agents and proapoptotie peptides to receptors located in tumor angiogenic vasculature resulted in a marked increase in therapeutic efficacy and a decrease in systemic toxicity in tumor bearing mouse models (Arap et al, 1998a, 1998b, Ellerby et al, 1999)

The methods described herein for identification of targeting peptides involve the in vivo administration of phage display libraries Various methods of phage display and methods for producing diverse populations of peptides are well known in the art For example, U.S. Pat. Nos. 5,223,409, 5,622,699, and 6,068,829, each of which is incorporated herein by reference in its entirety, disclose methods for preparing a phage library The phage display technique involves genetically manipulating bacteriophage so that small peptides can be expressed on their surface (Smith and Scott, 1985 and 1993) The potential range of applications for this technique is quite broad, and the past decade has seen considerable progress in the construction of phage-displayed peptide libraries and in the development of screening methods in which the libraries are used to isolate peptide ligands For example, the use of peptide libraries has made it possible to characterize interacting sites and receptor-ligand binding motifs within many proteins, such as antibodies involved in inflammatory reactions or integnns that mediate cellular adherence This method has also been used to identify novel peptide ligands that serve as leads to the development of peptidornimetic drugs or imaging agents (Arap et al, 1998a) In addition to peptides, larger protein domains such as single-chain antibodies can also be displayed on the surface of phage particles (Aran et al, 1998a)

D. Choice of Phage Display System

Previous in vivo selection studies performed in mice preferentially employed libraries of random peptides expressed as fusion proteins with the gene III capsule protein in the fUSE5 vector (Pasqualini and Ruoslahti, 1996) The number and diversity of individual clones present in a given library is a significant factor for the success of in vivo selection It is preferred to use primary libraries, which are less likely to have an over-representation of defective phage clones (Koivunen et al, 1999b) The preparation of a library should be optimized to between 10⁸-10⁹ transducing units (TU)/ml In certain embodiments, a bulk amplification strategy is applied between each round of selection

Phage libraries displaying linear, cyclic, or double cyclic peptides may be used within the scope of the present invention However, phage libraries displaying 3 to 10 random residues in a cyclic insert (CX₃₋₁₀C) are preferred, since single cyclic peptides tend to have a higher affinity for the target tissue or organ than linear peptides Libraries displaying double-cyclic peptides (such as CX₃C X₃CX₃C, Rojotte et al, 1998) have been successfully used However, the production of the cognate synthetic peptides, although possible, can be complex due to the multiple conformers with different disulfide bridge arrangements

### III. Targeted Delivery

Peptides that home to vasculature have been coupled to cytotoxic drugs or proapoptotic peptides to yield compounds that were more effective and less toxic than the parental compounds The present invention describes methods and compositions for the selective targeting of various tissues or organs

A “receptor” for a targeting peptide includes but is not limited to any molecule or macromolecular complex that binds to a targeting peptide Non-limiting examples of receptors include peptides, proteins, glycoproteins, lipoproteins, epitopes, lipids, carbohydrates, multi-molecular structures, and a specific conformation of one or more molecules In preferred embodiments, a “receptor” is a naturally occurring molecule or complex of molecules that is present on the surface of cells within a target tissue or organ More preferrably, a “receptor” is a naturally occurring molecule or complex of molecules that is present on or in a tissue, organ or vasculature thereof

In certain embodiments, therapeutic agents may be attached to a targeting peptide or fusion protein for selective delivery to, for example, leukemic cells or derivatives thereof Agents or factors suitable for use may include any chemical compound that induces apoptosis, cell death, cell stasis and/or anti-angiogenesis

A. Regulators of Programmed Cell Death

Apoptosis, or programmed cell death, is an essential process for normal embryonic development, maintaining homeostasis in adult tissues, and suppressing carcinogenesis (Kerr et al, 1972) The Bcl-2 family of proteins and ICE-like proteases have been demonstrated to be important regulators and effectors of apoptosis in other systems The Bcl 2 protein, discovered in association with follicular lymphoma, plays a prominent role in controlling apoptosis and enhancing cell survival in response to diverse apoptotic stimuli (Bakhshi et al, 1985, Cleary and Sklar, 1985, Cleary et al, 1986, Tsujimoto et al, 1985, Tsujimoto and Croce, 1986) The evolutionanly conserved Bcl 2 protein now is recognized to be a member of a family of related proteins, which can be categorized as death agonists or death antagonists

Subsequent to its discovery, it was shown that Bel 2 acts to suppress cell death triggered by a variety of stimuli Also, it now is apparent that there is a family of Bcl 2 cell death regulatory proteins that share in common structural and sequence homologies These different family members have been shown to either possess similar functions to Bcl 2 (e g, BclXL, BclW, BclS, Mcl-1, A1, Bfl-1) or counteract Bcl 2 function and promote cell death (e g, Bax, Bak, Bik, Bim, Bid, Bad, Harakiri)

Non-limiting examples of pro-apoptosis agents contemplated within the scope of the present invention include gramicidin, magainin, mellitin, defensin, cecropin, (KLAKLAK)₂ (SEQ ID NO 1), (KLAKKLA)₂ (SEQ ID NO 2), (KAAKKAA)₂ (SEQ ID NO 3) or (KLGKKLG)₃ (SEQ ID NO 4)

B. Angiogenic inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, pachtaxel, accutin, angiostatin, cidofovir, vincristine, bleomycin, AGM-1470, platelet factor 4 or minocycline

Proliferation of some tumor or cancer cells rely heavily on extensive tumor vascularization, which accompanies cancer progression Thus, inhibition of new blood vessel formation with anti-angiogenic agents and targeted destruction of existing blood vessels have been introduced as an effective and relatively non-toxic approach to tumor treatment (Arap et al, 1998, Arap et al, 1998, Ellerby at al, 1999) A variety of anti-angiogenic agents and/or blood vessel inhibitors are known (e g, Folkman, 1997, Eliceiri and Cheresh, 2001)

C. Cytotoxic Agents

Chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof

Chemotherapeutic agents and methods of administration, dosages, etc are well known to those of skill in the art (see for example, the “Physicians Desk Reference”, Goodman & Gilman's “The Pharmacological Basis of Therapeutics” and in “Remington's Pharmaceutical Sciences” 15th ed, pp 1035-1038 and 1570-1580, each incorporated herein by reference in relevant parts), and may be combined with the invention in light of the disclosures herein Some variation in dosage will necessarily occur depending on the condition of the subject being treated The person responsible for administration will, in any event, determine the appropriate dose for the individual subject Examples of specific chemotherapeutic agents and dose regimes are also described herein Of course, all of these dosages and agents described herein are exemplary rather than limiting, and other doses or agents may be used by a skilled artisan for a specific patient or application Any dosage within these points, or range derivable therein is also expected to be of use in the invention

D. Alkylating agents

Alkylating agents are drugs that directly interact with genomic DNA to prevent cells from proliferating This category of chemotherapeutic drugs represents agents that affect all phases of the cell cycle, that is, they are not phase-specific An alkylating agent, may include, but is not limited to, a nitrogen mustard, an ethylenimene, a methylmelamine, an alkyl sulfonate, a nitrosourea or a triazines They include but are not limited to busulfan, chlorambucil, cisplatin, cyclophosphamide (cytoxan), dacarbazine, ifosfamide, mechlorethamine (mustargen), and melphalan

E. Antimetabolites

Antimetabolites disrupt DNA and RNA synthesis Unlike alkylating agents, they specifically influence the cell cycle during S phase Antimetabolites can be differentiated into various categories, such as folic acid analogs, pynmidine analogs and purine analogs and related inhibitory compounds Antimetabolites include but are not limited to, 5-fluorouracil (5-FU), cytarabine (Ara-C), fludarabine, gemcitabine, and methotrexate

F. Natural Products

Natural products generally refer to compounds originally isolated from a natural source, and identified as having a pharmacological activity Such compounds, analogs and derivatives thereof may be, isolated from a natural source, chemically synthesized or recombinantly produced by any technique known to those of skill in the art Natural products include such categories as mitotic inhibitors, antitumor antibiotics, enzymes and biological response modifiers

Mitotic inhibitors include plant alkaloids and other natural agents that can inhibit either protein synthesis required for cell division or mitosis They operate during a specific phase during the cell cycle Mitotic inhibitors include, for example, docetaxel, etoposide (VP 16), teniposide, paclitaxel, taxol, vinblastine, vincristine, and vinorelbine

Taxoids are a class of related compounds isolated from the bark of the ash tree, Taxus brevifolia Taxoids include but are not limited to compounds such as docetaxel and pachtaxel Pachtaxel binds to tubulin (at a site distinct from that used by the vinca alkaloids) and promotes the assembly of microtubules

Vinca alkaloids are a type of plant alkaloid identified to have pharmaceutical activity They include such compounds as vinblastine (VLB) and vincristine

G. Antibiotics

Certain antibiotics have both antimicrobial and cytotoxic activity These drugs also interfere with DNA by chemically inhibiting enzymes and mitosis or altering cellular membranes These agents are not phase specific so they work in all phases of the cell cycle Examples of cytotoxic antibiotics include, but are not limited to, bleomycin, dactinomycin, daunorubicin, doxorubicin (Adriamycin), phcamycin (mithramycin) and idarubicin

H. Miscellaneous Agents

Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin Platinum coordination complexes include such compounds as carboplatin and cisplatin (cis-DDP) An exemplary anthracenedione is mitoxantrone An exemplary substituted urea is hydroxyurea An exemplary methyl hydrazine derivative is procarbazine (N-methylhydrazine, MIH) These examples are not limiting and it is contemplated that any known cytotoxic, cytostatic or cytocidal agent may be attached to targeting peptides and administered to a targeted organ, tissue or cell type within the scope of the invention

I. Dosages

The skilled artisan is directed to “Remington's Pharmaceutical Sciences” 15th Edition, chapter 33, and in particular to pages 624-652 Some variation in dosage will necessarily occur depending on the condition of the subject being treated The person responsible for administration will, in any event, determine the appropriate dose for the individual subject Moreover, for human administration, preparations should meet sterility, pyrogenicity, and general safety and punty standards as required by the FDA Office of Biologics standards

### IV. Proteins and Peptides

In certain embodiments, the present invention concerns novel compositions compnsing at least one protein or peptide As used herein, a protein or peptide generally refers, but is not limited to, a protein of greater than about 200 amino acids, up to a full length sequence translated from a gene, a polypeptide of greater than about 100 amino acids, and/or a peptide of from about 3 to about 100 amino acids

In certain embodiments the size of at least one peptide may comprise, but is not limited to, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids In other embodiments the size of at least one protein may comprise, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues

As used herein, an “amino acid residue” refers to any naturally occurring amino acid, any amino acid derivative or any amino acid mimetic known in the art In certain embodiments, the residues of the protein or peptide are sequential, without any non-amino acid interrupting the sequence of amino acid residues In other embodiments, the sequence may comprise one or more non-amino acid moieties In particular embodiments, the sequence of residues of the protein or peptide may be interrupted by one or more non-amino acid moieties Accordingly, the term protein or peptide encompasses amino acid sequences comprising at least one of the 20 common amino acids found in naturally occurring proteins, or at least one modified or unusual amino acid, including, but not limited to, 2 Aminoadipic acid (Aad), N Ethylasparagine (EtAsn), 3 Aminoadipic acid (Baad), Hydroxylysine (Hyl), βalanine, β Amino propionic acid (Bala), allo Hydroxylysine (AHy1), 2 Aminobutyric acid (Abu), 3 Hydroxyproline (3Hyp), 4 Aminobutyric acid (4Abu), 4 Hydroxyproline (4Hyp), 6 Aminocaproic acid (Acp), Isodesmosine (Ide), 2 Aminoheptanoic acid (Ahe), allo Isoleucine (AIle), 2 Aminoisobutyric acid (Aib), N Methylglycine (MeGly), 3 Aminoisobutyric acid (Baib), N Methylisoleucine (MeIle), 2 Aminopimelic acid (Apm), 6 N Methyllysine (MeLys), 2,4 Diaminobutyric acid (Dbu), N Methylvaline (MeVal), Desmosine (Des), Norvaline (Nva), 2,2′ Diaminopimelic acid (Dpm), Norleucine (Nle), 2,3 Diaminopropionic acid (Dpr), Ornithine (Orn), or N Ethylglycine (EtGly)

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides Coding regions for known genes may be amplified and/or expressed using the techniques disclosed herein or as would be know to those of ordinary skill in the art Alternatively, various commercial preparations of proteins, polypeptides and peptides are also known to those of skill in the art

A. Peptide Mimetics

Another embodiment for the preparation of molecule or compound according to the invention is the use of peptide mimetics that mimic characteristics of all or part of the peptides identified herein Mimetics are molecules that mimic elements of protein secondary structure (see, for example, Johnson et al, 1993, incorporated herein by reference) The underlying rationale behind the use of peptide mimetics is that the peptide backbone of proteins exists chiefly to orient amino acid side chains in such a way as to facilitate molecular interactions, such as those of antibody and antigen A peptide mimetic is expected to permit molecular interactions similar to the natural molecule These principles may be used to engineer second generation molecules having many of the natural properties of the targeting peptides disclosed herein, but with altered and even improved characteristics

B. Fusion Proteins

Other embodiments of the present invention concern fusion proteins These molecules generally have all or a substantial portion of a targeting peptide, linked at the N- or C-terminus or inserted within a known protein or peptide sequence, to all or a portion of a second polypeptide or protein For example, fusions may employ leader sequences from other species to permit the recombinant expression of a protein in a heterologous host Another useful fusion includes the addition of an immunologically active domain, such as an antibody epitope, to facilitate purification of the fusion protein Inclusion of a cleavage site at or near the fusion Junction will facilitate removal of the extraneous polypeptide after purification Other useful fusions include linking of functional domains, such as active sites from enzymes, glycosylation domains, cellular targeting signals or transmembrane regions In preferred embodiments, the fusion proteins of the instant invention comprise a targeting peptide linked to a therapeutic protein or peptide Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins These examples are not meant to be limiting and it is contemplated that within the scope of the present invention virtually any protein or peptide could be incorporated into a fusion protein comprising a targeting peptide identified by the methods of the invention

Methods of generating fusion proteins are well known to those of skill in the art Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein

C. Protein Purification

In certain embodiments a protein or peptide may be isolated or purified Protein purification techniques are well known to those of skill in the art These techniques involve, at one level, the homogenization and crude fractionation of cells, tissue or organ to polypeptide and non-polypeptide fractions The protein or peptide of interest may be further purified using chromatographic and electrophoretic techniques to achieve partial or complete purification (or purification to homogeneity) Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing An example of receptor protein purification by affinity chromatography is disclosed in U.S. Pat. No. 5,206,347, the entire text of which is incorporated herein by reference A particularly efficient method of purifying peptides is fast performance liquid chromatography (FPLC) or even high performance liquid chromatography (HPLC)

A purified protein or peptide is intended to refer to a composition, isolatable from other components, wherein the protein or peptide is purified to any degree relative to its naturally-obtainable state An isolated or purified protein or peptide, therefore, also refers to a protein or peptide free from the environment in which it may naturally occur Generally, “purified” will refer to a protein or peptide composition that has been subjected to fractionation to remove various other components, and which composition substantially retains its expressed biological activity Where the term “substantially purified” is used, this designation will refer to a composition in which the protein or peptide forms the major component of the composition, such as constituting about 50%, about 60%, about 70%, about 80%, about 90%, about 95%, or more of the protein or peptide in the composition

Various methods for quantifying the degree of punfication of the protein or peptide are known to those of skill in the art in light of the present disclosure These include, for example, determining the specific activity of an active fraction, or assessing the amount of protein or peptide within a fraction by SDS/PAGE analysis A preferred method for assessing the purity of a fraction is to calculate the specific activity of the fraction, to compare it to the specific activity of the initial extract, and to thus calculate the degree of purity therein, assessed by a “-fold punfication number” The actual units used to represent the amount of activity will, of course, be dependent upon the particular assay technique chosen to follow the purification, and whether or not the expressed protein or peptide exhibits a detectable activity

Various techniques suitable for use in protein purification are well known to those of skill in the art These Include, for example, precipitation with ammonium sulphate, PEG, antibodies and the like, by heat denaturation, centrifugation, chromatography steps such as ion exchange, gel filtration, reverse phase, hydroxylapatite, affinity chromatography, isoelectric focusing, gel electrophoresis, alone or in combination with these and other techniques As is generally known in the art, it is believed that the order of conducting the various purification steps may be changed, or that certain steps may be omitted, and still result in a suitable method for the preparation of a substantially purified protein or peptide

There is no general requirement that the protein or peptide always be provided in their most purified state Indeed, it is contemplated that less substantially purified products will have utility in certain embodiments Partial purification may be accomplished by using fewer purification steps in combination, or by utilizing different forms of the same general purification scheme For example, it is appreciated that a cation-exchange column chromatography performed utilizing an HPLC apparatus will generally result in a greater “-fold” purification than the same technique utilizing some other chromatography systems Methods exhibiting a lower degree of relative purification may have advantages in total recovery of protein product, or in maintaining the activity of an expressed protein

Affinity chromatography is a chromatographic procedure that relies on the specific affinity between a substance to be isolated and a molecule to which it can specifically bind, for example a receptor-ligand type of interaction The column material is synthesized by covalently coupling one of the binding partners to an insoluble matrix The column material is then able to specifically adsorb the substance from the solution Elution occurs by changing the conditions to those in which binding will not occur (e g, altered pH, ionic strength, and temperature) The matrix should be a substance that itself does not adsorb molecules to any significant extent and that has a broad range of chemical, physical and thermal stability The ligand should be coupled in such a way as to not affect its binding properties The ligand should also provide relatively tight binding And it should be possible to elute the substance without destroying the sample or the ligand

D. Synthetic Peptides

Because of their relatively small size, some exemplary targeting peptides of the invention can be synthesized in solution or on a solid support in accordance with conventional techniques Various automatic synthesizers are commercially available and can be used in accordance with known protocols (see, for example, Stewart and Young, 1984, Tam et al, 1983, Merrifield, 1986, or Barany and Merrifield, 1979, each incorporated herein by reference) Short peptide sequences, usually from about 6 up to about 35 to 50 amino acids, can be readily synthesized by such methods Alternatively, recombinant DNA technology may be employed wherein a nucleotide sequence which encodes a peptide of the invention is inserted into an expression vector, transformed or transfected into an appropriate host cell, and cultivated under conditions suitable for expression

E. Antibodies

In certain embodiments, it may be desirable to make antibodies against the identified targeting peptides or their receptors The appropriate targeting peptide or receptor, or portions thereof, may be coupled, bonded, bound, conjugated, or chemically-linked to one or more agents via linkers, polylinkers, or derivatized amino acids This may be performed such that a bispecific or multivalent composition or vaccine is produced It is further envisioned that the methods used in the preparation of these compositions are familiar to those of skill in the art and should be suitable for administration to humans, i e, pharmaceutically acceptable Preferred agents are the carriers are keyhole limpet hemocyanin (KLH) or bovine serum albumin (BSA)

The term “antibody” is used to refer to any antibody like molecule that has an antigen binding region, and includes antibody fragments such as Fab′, Fab, F(ab′)2, single domain antibodies (DABs), Fv, scFv (single chain Fv), and the like Techniques for preparing and using various antibody based constructs and fragments are well known in the art Means for preparing and characterizing antibodies are also well known in the art (See, e g, Harlow and Lane, 1988, incorporated herein by reference)

F. Cytokines and Chemokines

In certain embodiments, it may be desirable to couple specific bioactive agents to one or more targeting peptides for targeted delivery to a tissue, an organ, or vasculature thereof Such agents include, but are not limited to, cytokines, chemokines, pro-apoptosis factors and anti-angiogenic factors The term “cytokine” is a generic term for proteins released by one cell population that act on another cell as intercellular mediators

Examples of such cytokines are lymphokines, monokines, growth factors and traditional polypeptide hormones Included among the cytokines are growth hormones such as human growth hormone, N-methionyl human growth hormone, and bovine growth hormone, parathyroid hormone, thyroxine, insulin, proinsulin, relaxin, prorelaxin, glycoprotein hormones such as follicle stimulating hormone (FSH), thyroid stimulating hormone (TSH), and luteinizing hormone (LH), hepatic growth factor, prostaglandin, fibroblast growth factor, prolactin, placental lactogen, OB protein, tumor necrosis factor-α and β, mullerian-inhibiting substance, mouse gonadotropin-associated peptide, inhibin, activin, vascular endothelial growth factor, integrin, thrombopoietin (TPO), nerve growth factors such as NGF-β, platelet-growth factor, transforming growth factors (TGFs) such as TGF-α and TGF-β, insulin-like growth factor-I and -II, erythropoietin (EPO), osteoinductive factors, interferons such as interferon-α, -β, and -γ, colony stimulating factors (CSFs) such as macrophage-CSF (M-CSF), granulocyte-macrophage-CSF (GM-CSF), and granulocyte-CSF (G-CSF), interleukins (ILs) such as IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor (TNF) and lymphotoxin (LT) As used herein, the term cytokine includes proteins from natural sources or from recombinant cell culture and biologically active equivalents of the native sequence cytokines

Chemokines generally act as chemoattractants to recruit immune effector cells to the site of chemokine expression It may be advantageous to express a particular chemokme gene in combination with, for example, a cytokine gene, to enhance the recruitment of other immune system components to the site of treatment Chemokines include, but are not limited to, RANTES, MCAF, MIP1-alpha, MIP1-Beta, and IP-10 The skilled artisan will recognize that certain cytokines are also known to have chemoattractant effects and could also be classified under the term chemokines

G. Imaging Agents and Radioisotopes

In certain embodiments, the claimed peptides or proteins of the present invention may be attached to imaging agents of use for imaging and diagnosis of various diseased tissues or organs Many appropriate imaging agents are known in the art, as are methods for their attachment to proteins or peptides (see, e g, U.S. Pat. Nos. 5,021,236 and 4,472,509, both of which are incorporated herein by reference) Certain attachment methods involve the use of a metal chelate complex employing, for example, an organic chelating agent such a DTPA attached to the protein or peptide (U.S. Pat. No. 4,472,509) Proteins or peptides also may be reacted with an enzyme in the presence of a coupling agent such as glutaraldehyde or periodate Conjugates with fluorescein markers are prepared in the presence of these coupling agents or by reaction with an isothiocyanate

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred Ions useful in other contexts, such as X ray imaging, include but are not limited to lanthanum (III), gold (III), lead (II), and especially bismuth (III)

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium^{90 125}I is often being preferred for use in certain embodiments, and technicium^{99m} and indium¹¹¹ are also often preferred due to their low energy and suitability for long range detection

Radioactively labeled proteins or peptides of the present invention may be produced according to well known methods in the art For instance, they can be iodinated by contact with sodium or potassium iodide and a chemical oxidizing agent such as sodium hypochlorite, or an enzymatic oxidizing agent, such as lactoperoxidase Proteins or peptides according to the invention may be labeled with technetium^{99m} by ligand exchange process, for example, by reducing pertechnate with stannous solution, chelating the reduced technetium onto a Sephadex column and applying the peptide to this column or by direct labeling techniques, e g, by incubating pertechnate, a reducing agent such as SNCl₂, a buffer solution such as sodium potassium phthalate solution, and the peptide Intermediary functional groups that are often used to bind radioisotopes that exist as metallic ions to peptides are diethylenetriaminepenta-acetic acid (DTPA) and ethylene diaminetetra-acetic acid (EDTA) Also contemplated for use are fluorescent labels, including rhodamine, fluorescein isothiocyanate and renographin

In certain embodiments, the claimed proteins or peptides may he linked to a secondary binding ligand or to an enzyme (an enzyme tag) that will generate a colored product upon contact with a chromogenic substrate Examples of suitable enzymes include urease, alkaline phosphatase, (horseradish) hydrogen peroxidase and glucose oxidase Preferred secondary binding ligands are biotin and avidin or streptavidin compounds The use of such labels is well known to those of skill in the art in light and is described, for example, in U.S. Pat. Nos. 3,817,837, 3,850,752, 3,939,350, 3,996,345, 4,277,437, 4,275,149 and 4,366,241, each incorporated herein by reference

H. Cross-Linkers

Bifunctional cross-linking reagents have been extensively used for a variety of purposes including preparation of affinity matrices, modification and stabilization of diverse structures, identification of ligand and receptor binding sites, and structural studies Homobifunctional reagents that carry two identical functional groups proved to be highly efficient in inducing cross-linking between identical and different macromolecules or subunits of a macromolecule, and linking of polypeptide ligands to their specific binding sites Heterobifunctional reagents contain two different functional groups By taking advantage of the differential reactivities of the two different functional groups, cross-linking can be controlled both selectively and sequentially The bifunctional cross-linking reagents can he divided according to the specificity of their functional groups, e g, amino, sulfhydryl, guanidino, indole, carboxyl specific groups Of these, reagents directed to free amino groups have become especially popular because of their commercial availability, ease of synthesis and the mild reaction conditions under which they can be applied A majonty of heterobifunctional cross-linking reagents contains a primary amine-reactive group and a thiol-reactive group

Exemplary methods for cross-linking ligands to liposomes are described in U.S. Pat. Nos. 5,603,872 and 5,401,511, each specifically incorporated herein by reference in its entirety Various ligands can be covalently bound to liposomal surfaces through the cross-linking of amine residues Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures The inclusion of PE in the liposome provides an active functional residue, a primary amine, on the liposomal surface for cross-linking purposes Ligands such as epidermal growth factor (EGF) have been successfully linked with PE-liposomes Ligands are bound covalently to discrete sites on the liposome surfaces The number and surface density of these sites are dictated by the liposome formulation and the liposome type The liposomal surfaces may also have sites for non-covalent association To form covalent conjugates of ligands and liposomes, cross-linking reagents have been studied for effectiveness and biocompatibility Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC) Through the complex chemistry of cross-linking, linkage of the amine residues of the recognizing substance and liposomes is established

In another example, heterobifunctional cross-linking reagents and methods of using the cross-linking reagents are described (U.S. Pat. No. 5,889,155, specifically incorporated herein by reference in its entirety) The cross-linking reagents combine a nucleophilic hydrazide residue with an electrophilic maleimide residue, allowing coupling in one example, of aldehydes to free thiols The cross-linking reagent can be modified to cross-link various functional groups

### V. Nucleic Acids

Nucleic acids according to the present invention may encode a targeting peptide, a receptor protein, a fusion protein, or other protein or peptide The nucleic acid may be derived from genomic DNA, complementary DNA (cDNA) or synthetic DNA Where incorporation into an expression vector is desired, the nucleic acid may also comprise a natural intron or an intron derived from another gene Such engineered molecules are sometime referred to as “mini-genes ”

A “nucleic acid” as used herein includes single-stranded and double-stranded molecules, as well as DNA, RNA, chemically modified nucleic acids and nucleic acid analogs It is contemplated that a nucleic acid within the scope of the present invention may be of almost any size, determined in part by the length of the encoded protein or peptide

It is contemplated that targeting peptides, fusion proteins and receptors may be encoded by any nucleic acid sequence that encodes the appropriate amino acid sequence The design and production of nucleic acids encoding a desired amino acid sequence is well known to those of skill in the art, using standardized codon tables In preferred embodiments, the codons selected for encoding each amino acid may be modified to optimize expression of the nucleic acid in the host cell of interest Codon preferences for vanous species of host cell are well known in the art

In addition to nucleic acids encoding the desired peptide or protein, the present invention encompasses complementary nucleic acids that hybridize under high stringency conditions with such coding nucleic acid sequences High stringency conditions for nucleic acid hybridization are well known in the art For example, conditions may comprise low salt and/or high temperature conditions, such as provided by about 0 02 M to about 0 15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture

A. Vectors for Cloning, Gene Transfer and Expression

In certain embodiments expression vectors are employed to express the targeting peptide or fusion protein, which can then be purified and used In other embodiments, the expression vectors are used in gene therapy Expression requires that appropriate signals be provided in the vectors, and which include various regulatory elements, such as enhancers/promoters from both viral and mammalian sources that drive expression of the genes of interest in host cells Elements designed to optimize messenger RNA stability and translatability in host cells also are known

### 1. Regulatory Elements

The terms “expression construct” or “expression vector” are meant to include any type of genetic construct containing a nucleic acid coding for a gene product in which part or all of the nucleic acid coding sequence is capable of being transcribed In preferred embodiments, the nucleic acid encoding a gene product is under transcriptional control of a promoter A “promoter” refers to a DNA sequence recognized by the synthetic machinery of the cell, or introduced synthetic machinery, required to initiate the specific transcription of a gene The phrase “under transcriptional control” means that the promoter is in the correct location and orientation in relation to the nucleic acid to control RNA polymerase initiation and expression of the gene

The particular promoter employed to control the expression of a nucleic acid sequence of interest is not believed to be important, so long as it is capable of directing the expression of the nucleic acid in the targeted cell Thus, where a human cell is targeted, it is preferable to position the nucleic acid coding region adjacent and under the control of a promoter that transcriptionally active in human cells Generally speaking, such a promoter might Include either a human or viral promoter

In various embodiments, the human cytomegalovirus (CMV) immediate early gene promoter, the SV40 early promoter, the Rouse sarcoma virus long terminal repeat, rat insulin promoter, and glyceraldehyde-3-phosphate dehydrogenase promoter can be used to obtain high-level expression of the coding sequence of interest The use of other viral or mammalian cellular or bacterial phage promoters that are well-known in the art to achieve expression of a coding sequence of interest is contemplated as well, provided that the levels of expression are sufficient for a given purpose

Where a cDNA insert is employed, one will typically include a polyadenylation signal to effect proper polyadenylation of the gene transcript The nature of the polyadenylation signal is not believed to be crucial to the successful practice of the invention, and any such sequence may be employed, such as human growth hormone and SV40 polyadenylation signals Also contemplated as an element of the expression construct is a terminator These elements can serve to enhance message levels and to minimize read through from the construct into other sequences

### 2. Selectable Markers

In certain embodiments of the invention, the cells containing nucleic acid constructs of the present invention may be identified in vitro or in vivo by including a marker in the expression construct Such markers would confer an identifiable change to the cell permitting easy identification of cells containing the expression construct Usually the inclusion of a drug selection marker aids in cloning and in the selection of transformants For example, genes that confer resistance to neomycin, puromycin, hygromycin, DHFR, GPT, zeocin, and histidinol are useful selectable markers Alternatively, enzymes such as herpes simplex virus thymidine kinase (tk) or chloramphenicol acetyltransferase (CAT) may be employed Immunologic markers also can be employed The selectable marker employed is not believed to be important, so long as it is capable of being expressed simultaneously with the nucleic acid encoding a gene product Further examples of selectable markers are well known to one of skill in the art

### 3. Delivery of Expression Vectors

There are a number of ways in which expression vectors may introduced into cells In certain embodiments of the invention, the expression construct comprises a virus or engineered construct derived from a viral genome The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988, Nicolas and Rubinstein, 1988, Baichwal and Sugden, 1986, Temin, 1986) Preferred gene therapy vectors are generally viral vectors

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct A preferred means of purifying the vector involves the use of buoyant density gradients, such as cesium chloride gradient centrifugation

DNA viruses used as gene vectors include the papovaviruses (e g, simian virus 40, bovine papilloma virus, and polyoma) (Ridgeway, 1988, Baichwal and Sugden, 1986) and adenoviruses (Ridgeway, 1988, Baichwal and Sugden, 1986)

One of the preferred methods for in vivo delivery involves the use of an adenovirus expression vector Although adenovirus vectors are known to have a low capacity for integration into genomic DNA, this feature is counterbalanced by the high efficiency of gene transfer afforded by these vectors

Generation and propagation of adenovirus vectors that are replication deficient depend on a unique helper cell line, designated 293, which is transformed from human embryonic kidney cells by Ad5 DNA fragments and constitutively expresses E1 proteins (Graham et al, 1977) Since the E3 region is dispensable from the adenovirus genome (Jones and Shenk, 1978), the current adenovirus vectors, with the help of 293 cells, carry foreign DNA in either the E1, the E3, or both regions (Graham and Prevec, 1991)

Helper cell lines may be derived from human cells such as human embryonic kidney cells, muscle cells, hematopoietic cells or human embryonic mesenchymal or epithelial cells Alternatively, the helper cells may be denved from the cells of other mammalian species that are permissive for human adenovirus Such cells include, for example, Vero cells or other monkey embryonic mesenchymal or epithelial cells As discussed, the preferred helper cell line is 293 Racher et al (1995) disclose improved methods for culturing 293 cells and propagating adenovirus

Adenovirus vectors have been used in eukaryotic gene expression (Levrero at al, 1991, Gomez-Foix et al, 1992) and vaccine development (Grunhaus and Horwitz, 1992, Graham and Prevec, 1991) Animal studies have suggested that recombinant adenovirus could be used for gene therapy (Stratford-Perricaudet and Perricaudet, 1991, Stratford-Perricaudet at al, 1990, Rich at al, 1993) Studies in administering recombinant adenovirus to different tissues include tracheal instillation (Rosenfeld at al, 1991, Rosenfeld at al, 1992), muscle injection (Ragot at al, 1993), peripheral intravenous injections (Herz and Gerard, 1993) and stereotactic innoculation into the brain (Le Gal La Salle et al, 1993)

Other gene transfer vectors may be constructed from retroviruses (Coffin, 1990) The retroviral genome contains three genes, gag, pol, and env that code for capsid proteins, polymerase enzyme, and envelope components, respectively A sequence found upstream from the gag gene contains a signal for packaging of the genome into virions Two long terminal repeat (LTR) sequences are present at the 5′ and 3′ ends of the viral genome These contain strong promoter and enhancer sequences, and also are required for integration in the host cell genome (Coffin, 1990)

In order to construct a retroviral vector, a nucleic acid encoding a protein of interest is inserted into the viral genome in the place of certain viral sequences to produce a virus that is replication-defective In order to produce virions, a packaging cell line containing the gag, poi, and env genes, but without the LTR and packaging components, is constructed (Mann et al, 1983) When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988, Temin, 1986, Mann et al, 1983) The media containing the recombinant retroviruses is then collected, optionally concentrated, and used for gene transfer Retroviral vectors are capable of infecting a broad variety of cell types However, integration and stable expression require the division of host cells (Paskind et al, 1975)

Other viral vectors may be employed as expression constructs Vectors derived from viruses such as vaccinia virus (Ridgeway, 1988, Baichwal and Sugden, 1986, Coupar et al, 1988), adeno-associated virus (AAV) (Ridgeway, 1988, Baichwal and Sugden, 1986, Hermonat and Muzycska, 1984), and herpes viruses may be employed They offer several attractive features for various mammalian cells (Friedmann, 1989, Ridgeway, 1988, Baichwal and Sugden, 1986, Coupar et al, 1988, Horwich et al, 1990)

Several non-viral methods for the transfer of expression constructs into cultured mammalian cells also are contemplated by the present invention These include calcium phosphate precipitation (Graham and van der Eb, 1973, Chen and Okayama, 1987, Rippe et al, 1990, DEAE dextran (Gopal, at al, 1985), electroporation (Tur-Kaspa et al, 1986, Potter et al, 1984), direct microinjection, DNA-loaded liposomes and lipofectamine-DNA complexes, cell sonication, gene bombardment using high velocity microprojectiles, and receptor-mediated transfection (Wu and Wu, 1987, Wu and Wu, 1988) Some of these techniques may be successfully adapted for in vivo or ex vivo use

In a further embodiment of the invention, the expression construct may be entrapped in a liposome Liposome-mediated nucleic acid delivery and expression of foreign DNA in vitro has been very successful Wong et at (1980) demonstrates the feasibility of liposome-mediated delivery and expression of foreign DNA in cultured chick embryo, HeLa, and hepatoma cells Nicolau et al (1987) accomplished successful liposome-mediated gene transfer in rats after intravenous injection

### VI. Pharmaceutical Compositions

Where clinical applications are contemplated, it may be necessary to prepare pharmaceutical compositions—expression vectors, virus stocks, proteins, antibodies and drugs—in a form appropriate for the intended application Generally, this will entail preparing compositions that are essentially free of impurities that could be harmful to humans or animals

One generally will desire to employ appropriate salts and buffers to render delivery vectors stable and allow for uptake by target cells Buffers also are employed when recombinant cells are introduced into a patient Aqueous compositions of the present invention may comprise an effective amount of a protein, peptide, antibody, fusion protein, recombinant phage and/or expression vector, dissolved or dispersed in a pharmaceutically acceptable carrier or aqueous medium The phrase “pharmaceutically or pharmacologically acceptable” refers to molecular entities and compositions that do not produce adverse, allergic, or other untoward reactions when administered to an animal or a human As used herein, “pharmaceutically acceptable carrier” includes any and all solvents, dispersion media, coatings, antibacterial and antifungal agents, isotonic and absorption delaying agents and the like The use of such media and agents for pharmaceutically active substances is well known in the art Except insofar as any conventional media or agent is incompatible with the proteins or peptides of the present invention, its use in therapeutic compositions is contemplated Supplementary active ingredients also can be incorporated into the compositions

The active compositions of the present invention may include classic pharmaceutical preparations Administration of these compositions according to the present invention are via any common route so long as the target tissue is available via that route This includes oral, nasal, buccal, rectal, vaginal or topical Alternatively, administration may be by orthotopic, intradermal, subcutaneous, intramuscular, intraperitoneal, intraarterial or intravenous injection Such compositions normally would be administered as pharmaceutically acceptable compositions, described supra

The pharmaceutical forms suitable for injectable use include sterile aqueous solutions or dispersions and sterile powders for the extemporaneous preparation of sterile injectable solutions or dispersions In all cases the form must be sterile and must be fluid to the extent that easy syringability exists It must be stable under the conditions of manufacture and storage and must be preserved against the contaminating action of microorganisms, such as bacteria and fungi The carrier can be a solvent or dispersion medium containing, for example, water, ethanol, polyol (for example, glycerol, propylene glycol, and liquid polyethylene glycol, and the like), suitable mixtures thereof, and vegetable oils The proper fluidity can be maintained, for example, by the use of a coating, such as lecithin, by the maintenance of the required particle size in the case of dispersion and by the use of surfactants The prevention of the action of microorganisms can be brought about by various antibacterial and antifungal agents, for example, parabens, chlorobutanol, phenol, sorbic acid, thimerosal, and the like In many cases, it is preferable to include isotonic agents, for example, sugars or sodium chloride Prolonged absorption of the injectable compositions can be brought about by the use in the compositions of agents delaying absorption, for example, aluminum monostearate and gelatin

Sterile injectable solutions are prepared by incorporating the active compounds in the required amount in the appropriate solvent with various other ingredients enumerated above, as required, followed by filtered sterilization Generally, dispersions are prepared by incorporating the various sterilized active ingredients into a sterile vehicle which contains the basic dispersion medium and the required other ingredients from those enumerated above In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof

### EXAMPLES

The following examples are included to demonstrate preferred embodiments of the invention It should be appreciated by those of skill in the art that the techniques disclosed in the examples which follow represent techniques discovered by the inventors to function well in the practice of the invention, and thus can be considered to constitute preferred modes for its practice However, those of skill in the art should, in light of the present disclosure, appreciate that many changes can be made in the specific embodiments which are disclosed and still obtain a like or similar result without departing from the spirit and scope of the invention

### Example 1

### Synchronous Multiple Organ Peptide Selection

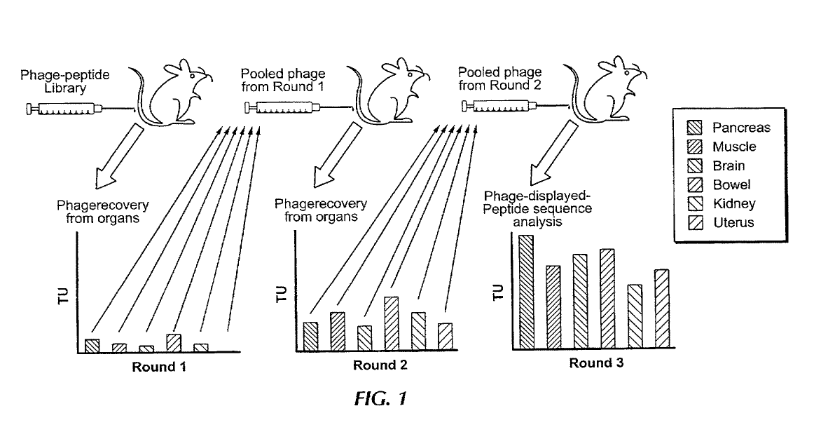

To establish the experimental framework for synchronous screening for peptides selectively homing to “n” given organs in the mouse model, a cyclic random phage display peptide library CX₇C (C, cysteine, X, any residue) was screened Six exemplary tissues or organs were processed muscle, intestine, uterus, kidney, pancreas, and brain (FIG. 1) Three rounds of library selection were performed based on the previously described methodology (Pasqualini et al 2000), but without whole-body perfusion of the vasculature (Paqualini and Ruoslahti, 1996), which was skipped in order to simulate screening conditions used in patients (Arap et al, 2002) In each round, peptide-displaying phage were isolated from target organs, amplified, and pooled for the next round of selection Given that peptide-displaying phage homing to each individual organ are likely to segregate independently, it was reasoned that the final round of selection would always yield peptides selectively localizing to a given target tissue To prove this hypothesis, peptide-encoding inserts from recovered phage clones were evaluated after each round of selection for each of the “n” organs targeted simultaneously

A. Materials and Methods

Synchronous screening of phage libraries in vivo C57B1/6 female mice were injected intravenously (iv) with 10¹⁰ transducing units (TU) of previously described (Pasqualini et al, 2001) library CX₇C (round 1) or a mixture (10⁹ TU per organ) of amplified phage recovered from each of the organs studied (rounds 2 and 3) For each round, phage were allowed to circulate for 15 min prior to organ recovery (without heart perfusion) After each round of selection, phage peptide-coding inserts were sequenced as described (Pasqualina et al, 2001), amplified for each organ individually, and subsequently pooled for the next round of in vivo selection

Statistical analysis of selected peptide motifs Calculation of tripeptide motif frequencies in CX₇C peptides encountered in each target tissue (in both directions) was done by using a character pattern recognition program based on SAS (version 8 1 2, SAS Institute) and Perl (version 5 8 1), as described (Arap et al, 2002) To identify tripeptides progressively enriched from round 1 to round 3 of panning, a Bayesian Beta/Binomial model was implemented by estimation of the posterior probability distribution for each tripeptide (Lee et al, 2003, Carlin and Sargent, 1996), posterior distributions for the proportion of each tripeptide in rounds 1 through 3 were calculated by using Spins (version 6) To determine the selectivity of tripeptide motif distribution in tissues, a Fisher's exact test (one-tailed) was used to calculate the P-values for the count of each tripeptide in a target tissue, as compared with its count within the 2,210 tripeptides of the unselected library (Table 1B) or the combined tripeptides from the other five tissues (Table 1C) Statistical uncertainty was further assessed by Monte Carlo simulations based on an established algorithm (Gelman and Rubin, 1996, Zhang et al, 1997) Using MATLAB, all tripeptide sequences (unselected library and selected for each organ) were pooled, and the combined tripeptide pool was distributed in 1,000 simulations into permutated groups corresponding in size to those analyzed in Table 1 for each organ and the preselection library For each round, Fisher's exact test was performed on the 1,000 scrambled datasets, and distributions of the 50 lowest P-values generated in each simulated test were compared with the distribution of 50 lowest P-values from the actual experimental data (the most significant of which are shown in Table 1 B)

High-throughput identification of peptide-mimicked proteins To facilitate large-scale peptide sequence analysis, an interactive peptide sequence management database was constructed based on MySQL Web-based peptide sequence retneval and management software based on Common Gateway Interface (CGI) and Perl was created, and integrated with the statistical analysis software To identify candidate cellular proteins mimicked by selected peptides, the database was consolidated with on-line ClustlW software (www ebi ac uk/clustalw/) to identify extended (4-7 residue long) motifs shared among multiple peptides homing to a specific tissue BLAST (www ncbi nlm nih gov/BLAST/) was used to identify proteins mimicked by the extended homing motifs by screening batches of ClustlW-identified peptide motifs against sequences contained in on-line non-redundant databases of mouse proteins To identify PRLR ligand-matching motifs among phage-displayed pancreas-homing peptides binding to PRLR, a software was codified in Perl 5 8 1 and run against ClustlW-aligned protein sequences for sheep PL (oPL), and mouse mPL-1 and mPRL Each seven-mer peptide sequence was aligned in each orientation against the protein sequences from N- to C-terminus in one-residue shifts The peptide-protein similarity scores for each residue were calculated based on a BLOSUM62 matrix modified to identify peptide matches of four or more residues in any position being identical to the corresponding amino acid positions in any of the three PRL homologues aligned

Phage peptide binding to PRLR To identify peptides binding to PRLR, phage clones isolated from the pancreas in rounds 2 and 3 of the screening were individually amplified and pooled For panning on immobilized PRLR, 10⁹ TU of the mixed phage clones were incubated overnight at 4° C. with 10 mg of purified recombinant rabbit PRLR (Protein Laboratories Rehovot, Israel), or BSA control, immobilized on plastic Unbound phage were extensively washed off with PBS, and then the bound phage were recovered by infecting host K91 E coli directly on the plate For panning on cell surface-expressed PRLR, COS-1 cells (ATCC) were transiently transfected with pECE-PRLR as described (Wang et al, 1997) and subjected to biopanning with the amplified pancreas-isolated phage using the BRASIL protocol (Giordano et al, 2001) In each biopanning, bound phage were selected for tetracycline resistance, quantified by infecting host K91 E coli and sequenced Single amino-acid substitutions and reversal of the PRL-matching motif displayed on phage was performed by PCR-directed mutagenesis of the peptide-coding insert (TGTCGCGTGGCGAGCGTGCTGCCGTGT)(SEQ ID NO 33) and cloning it into the fUSE5 vector (Smith and Scott, 1993) Phage displaying forward (CRVASVLPC)(SEQ ID NO 30), reverse CPLVSAVRC(SEQ ID NO 34), and the alanine point mutants were titered in-parallel with insertless fd-tet phage and tested for binding to COS-1 cells transfected with PRLR by using the BRASIL method (Giordano et al, 2001) COS-1 cells not transfected with PRLR served as a negative control

Immunolocalization Immunofluorescent detection of PRLR expression in COS-1 cells was performed by using anti-PRLR antibody MA1-610 (Affinity Bioreagents) diluted to 20 μg/ml and a secondary FITC-conjugated goat anti-mouse antibody F-0257 (Sigma) at 1 100 dilution For validating phage binding and internalization into COS-1 cells transfected with PRLR, 10⁹ TU of phage displaying PRLR-binding or mutant peptides were subjected to cell binding and internalization as described (Zurita et al, 2004) Immunodetection of cell-associated phage was performed with anti-fd antibody B-7786 (Sigma) at 1 500 dilution and a secondary Cy3-conjugated donkey anti-rabbit antibody 711-165-152 (Jackson) at 3 μg/ml For phage-peptide immunolocalization in situ, 10¹⁰ TU of iv-injected phage were let circulate for 5 mm Immunohistochemistry on formalin-fixed, paraffin-embedded mouse tissue sections was performed as described (Paqualini et al, 2001, Arap et al, 2002) by using anti-fd antibody B-7786 at 1 1,000 dilution and the LSAB+ peroxidase kit (DAKO)

TABLE 1
Peptide motifs homing to mouse tissues
Table 1A - Tripeptides progressively enriched in Rounds 1-3

| | | | | Posterior |
|---|---|---|---|---|
| Target organ/ | Motif frequency (%) | | | probability fold over |
| motif | ROUND 1 | ROUND 2 | ROUND 3 | baseline |
| Muscle | | | | |
| RSG | 2 2 | 2 4 | 5 6 | 10 0 |
| SGA | 0 0 | 1 2 | 5 6 | 7 0 |
| AIG | 0 0 | 0 0 | 4 4 | 5 0 |
| IGS | 0 0 | 1 2 | 5 6 | 7 0 |
| GSF | 0 0 | 1 2 | 5 6 | 7 0 |
| AGG | 1 1 | 1 2 | 4 4 | 7 0 |
| ASR | 0 0 | 0 0 | 3 3 | 4 0 |
| DFS | 0 0 | 0 0 | 4 4 | 5 0 |
| DGT | 0 0 | 0 0 | 3 3 | 4 0 |
| DTG | 0 0 | 0 0 | 3 3 | 4 0 |
| FRS | 0 0 | 1 2 | 3 3 | 5 0 |
| GDT | 0 0 | 1 2 | 3 3 | 5 0 |
| GGT | 0 0 | 0 0 | 5 6 | 6 0 |
| GWS | 0 0 | 0 0 | 3 3 | 4 0 |
| IAY | 0 0 | 0 0 | 4 4 | 5 0 |
| RRS | 0 0 | 1 2 | 3 3 | 5 0 |
| SGV | 0 0 | 1 2 | 3 3 | 5 0 |
| Pancreas | | | | |
| LVS | 1 1 | 3 3 | 4 8 | 9 0 |
| VSS | 1 1 | 0 0 | 7 1 | 8 0 |
| WSG | 1 1 | 0 0 | 6 0 | 7 0 |
| GWR | 0 0 | 1 1 | 3 6 | 5 0 |
| GYN | 0 0 | 0 0 | 3 6 | 4 0 |
| LTR | 0 0 | 1 1 | 3 6 | 5 0 |
| TLV | 0 0 | 0 0 | 3 6 | 4 0 |
| Brain | | | | |
| LGG | 1 1 | 0 0 | 5 8 | 8 0 |
| RGF | 0 0 | 0 0 | 3 5 | 4 0 |
| ALG | 2 1 | 0 0 | 3 5 | 6 0 |
| LLS | 1 1 | 0 0 | 3 5 | 5 0 |
| Kidney | | | | |
| LGS | 1 1 | 2 3 | 4 4 | 8 0 |
| SLS | 1 1 | 1 1 | 4 4 | 7 0 |
| DRG | 0 0 | 0 0 | 3 3 | 4 0 |
| RRV | 0 0 | 0 0 | 3 3 | 4 0 |
| DSG | 0 0 | 1 1 | 3 3 | 5 0 |
| LRV | 0 0 | 1 1 | 3 3 | 5 0 |
| SRV | 1 1 | 1 1 | 4 4 | 7 0 |
| Uterus | | | | |
| GSS | 1 2 | 0 0 | 4 9 | 6 0 |
| LLG | 1 2 | 0 0 | 4 9 | 6 0 |
| GAA | 0 0 | 1 1 | 4 9 | 6 0 |
| GLL | 1 2 | 0 0 | 4 9 | 6 0 |
| ARG | 1 2 | 3 2 | 3 7 | 8 0 |
| GAS | 0 0 | 1 1 | 3 7 | 5 0 |
| GGL | 2 4 | 1 1 | 4 9 | 8 0 |
| GPS | 0 0 | 0 0 | 3 7 | 4 0 |
| Bowel | | | | |
| AGV | 0 0 | 0 0 | 4 7 | 5 0 |
| WRD | 0 0 | 0 0 | 4 7 | 5 0 |
| FGG | 0 0 | 0 0 | 4 7 | 5 0 |
| GGR | 1 1 | 0 0 | 7 1 | 8 0 |
| GRV | 0 0 | 1 2 | 3 5 | 5 0 |
| RWS | 0 0 | 0 0 | 3 5 | 4 0 |
| VGV | 0 0 | 0 0 | 3 5 | 4 0 |

| | | |
|---|---|---|
| Target organ/motif | Motif frequency (%) | P value |

Table 1B - Tripeptides selected (vs. unselected library)

| | | |
|---|---|---|
| Muscle | | |
| RSG | 5 6 | 0 0301 |
| SGA | 5 6 | 0 0301 |
| AIG | 4 4 | 0 0006 |
| IGS | 5 6 | 0 0037 |
| GSF | 5 6 | 0 0072 |
| AGG | 4 4 | 0 0261 |
| APA | 2 2 | 0 0253 |
| DFS | 4 4 | 0 0028 |
| GDT | 3 3 | 0 0040 |
| GGT | 5 6 | 0 0201 |
| IAY | 4 4 | 0 0006 |
| Pancreas | | |
| LVS | 4 8 | 0 0022 |
| VSS | 7 1 | 0 0006 |
| WSG | 60 | 0 0012 |
| FGV | 3 6 | 0 0033 |
| GYN | 3 6 | 0 0033 |
| LTR | 3 6 | 0 0118 |
| TLV | 3 6 | 0 0033 |
| Brain | | |
| LGG | 5 8 | 0 0204 |
| RGF | 3 5 | 0 0337 |
| DSY | 2 3 | 0 0174 |
| GFS | 2 3 | 0 0174 |
| GIW | 2 3 | 0 0174 |
| HGL | 2 3 | 0 0477 |
| Kidney | | |
| SLS | 4 4 | 0 0004 |
| DRG | 3 3 | 0 0239 |
| RRV | 3 3 | 0 0239 |
| SRV | 4 4 | 0 0186 |
| FLS | 2 2 | 0 0208 |
| Uterus | | |
| GSS | 4 9 | 0 0456 |
| LLG | 4 9 | 0 0308 |
| GAA | 4 9 | 0 0109 |
| GPS | 3 7 | 0 0246 |
| Bowel | | |
| AGV | 4 7 | 0 0719 |
| WRD | 4 7 | 0 0023 |
| FGG | 4 7 | 0 0005 |
| RWS | 3 5 | 0 0275 |
| VGV | 3 5 | 0 0124 |
| GVG | 3 5 | 0 0488 |

Table 1C - Tripeptides specific (vs other organs)

| | | |
|---|---|---|
| Muscle | | |
| RSG | 5 6 | 0 0351 |
| SGA | 5 6 | 0 0132 |
| AIG | 4 4 | 0 0242 |
| IGS | 5 6 | 0 0010 |
| GSF | 5 6 | 0 0030 |
| APA | 2 2 | 0 0333 |
| DFS | 4 4 | 0 0121 |
| GDT | 3 3 | 0 0210 |
| GGT | 5 6 | 0 0010 |
| IAY | 4 4 | 0 0011 |
| Pancreas | | |
| VSS | 7 1 | 0 0012 |
| WSG | 60 | 0 0021 |
| FGV | 3 6 | 0 0371 |
| GYN | 3 6 | 0 0169 |
| LTR | 3 6 | 0 0048 |
| TLV | 3 6 | 0 0371 |
| Brain | | |
| LGG | 5 8 | 0 0427 |
| RGF | 3 5 | 0 0250 |
| DSY | 2 3 | 0 0215 |
| GIW | 2 3 | 0 0215 |
| HGL | 2 3 | 0 0215 |
| ALG | 3 5 | 0 0446 |
| LLS | 3 5 | 0 0446 |
| Kidney | | |
| SLS | 4 4 | 0 0030 |
| DRG | 3 3 | 0 0151 |
| FLS | 2 2 | 0 0265 |
| Uterus | | |
| GPS | 3 7 | 0 0157 |
| GAA | 4 9 | 0 0322 |
| Bowel | | |
| AGV | 4 7 | 0 0344 |
| WRD | 4 7 | 0 0098 |
| FGG | 4 7 | 0 0199 |
| RWS | 3 5 | 0 0179 |

Table 1A Tripeptides progressively enriched in Rounds 1-3 using the Bayesian Beta/Binomial model, tripeptides were ranked according to posterior mean (shown are tripeptides with posterior probability fold change of x3 or more over baseline (posterior probability for tripeptides not isolated in any of the three rounds)
Table 1B tripeptide motifs contained in CX₇C peptides isolated in round 3 from target organs with frequency significantly higher than that observed in the unselected phage library (Fisher Exact test, one-tailed, P < 0 05)
Table 1C tripeptide motifs occurring in CX₇C peptides enriched in round 3 in a specific organ but not in other target organs analyzed (Fisher Exact test, one-tailed, P < 0 05) The P-value for the tripeptide LVS in the pancreas is 0 13

B. Results

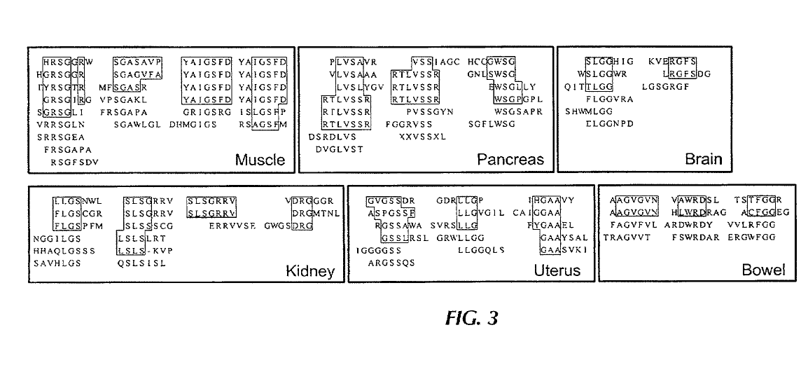

For validation of potentially specific organ-horning motifs, the inventors chose to focus on tripeptides that fulfilled statistical tests criteria (Table 1), however, the inventors also took into account whether more than a single tripeptide had homology to candidate ligands To determine if the tripeptides represented parts of longer motifs responsible for organ homing, the ClustalW software (www ebi ac uk/clustalw/) was applied Selected tripeptides led to identification of extended motifs shared among ligand peptides isolated from a given organ (FIG. 3)

Next, the inventors screened each of the extended motifs against a non-redundant database of mouse proteins (www ncbi nlm nih gov/BLAST/) to identify binding sites within proteins potentially mimicked by the motifs The inventors systematically analyzed similarities to extracellular signaling factors that regulate organ-dependent vascular growth or homeostasis and mapped 16 motifs to segments of such proteins, in some cases, several motifs capable of homing to an organ that mapped to different domains were found within a single protein (Table 2) Interestingly, for skeletal muscle and pancreas, homing tripeptides were mapped to various domains of different ligands sharing a receptor with a functional role in vascular biology, moreover, the inventors found more than one apparent peptide mimic for some ligands of this class (Table 2) For example, independent tripeptides homing to the muscle matched to different proteins known to interact with receptors of the Notch family Of such motifs, the tripeptides FSG and SGI were partially overlapping in the extended DFSGIA+ (SEQ ID NO 12) region of similarity to disintegrin family metalloproteinases that cleave Notch receptors (Brou et al, 2000) (Table 2) Moreover, the motifs GRSG+R (SEQ ID NO 13) and SGASAV (SEQ ID NO 14), matched to two different domains of the Jagged2-like protein that belongs to a family of Notch ligands (Linder et al, 2001) (Table 2) For the pancreas, the motifs LVSA (SEQ ID NO 18) and WSGL (SEQ ID NO 19) showed close similarity to different domains of placental lactogen (PL-I), whereas the motif SWSG (SEQ ID NO 32) (also containing the tripeptide WSG) matched to prolactin-like protein M (PLP-M)

Because both PL-I and PLP-M belong to the family of prolactin-like peptidic hormones, which have been shown to function in the pancreas during pregnancy (Weimers et al, 2003, Brelje et al, 2002, Freemark et al, 2002), the inventors attempted to find a receptor that could mediate homing of the placental lactogen-mimicking LVSA (SEQ ID NO 18) motif to the pancreas The inventors administered a CRVASVLPC (SEQ ID NO 30)-phage clone (displaying the reversed LVSA (SEQ ID NO 18)) intravenously (iv) into mice and examined its tissue distribution Immunohistochemical analysis of mouse tissues with an anti-phage antibody (Pasqualini et al, 2000) showed that CRVASVLPC (SEQ ID NO 30)-phage localized to pancreatic blood vessels and the islets of Langerhans, whereas a control muscle-homing CYAIGSFDC (SEQ ID NO 31)-phage clone was found predominantly in the vasculature of skeletal muscle Because prolactin receptor (prlR) is the only known receptor for placental lactogens(Weimers et al, 2003, Brelje et al, 2002, Freemark et al, 2002), the inventors proposed that LVSA (SEQ ID NO 18)-containing motifs may mimic placental lactogens by binding prlR in vivo To test this hypothesis, the inventors first showed that the accumulation of mouse prlR protein in the pancreatic blood vessels and the islet cells (Brelje et al, 2002) closely resembles the distribution of CRVASVLPC (SEQ ID NO 30)-phage

Next, the inventors directly demonstrated a protein-protein interaction between CRVASVLPC (SEQ ID NO 30)-phage and prlR Specifically, binding of CRVASVLPC (SEQ ID NO 30)-phage to COS-1 cells transfected with prlR was five times higher than background binding by controls such as muscle-homing phage or insertless phage Finally, the inventors co-localized bound CRVASVLPC (SEQ ID NO 30)-phage and prlR-expressing cells by using immunofluorescence on the same prlR-transfected cells In contrast, no co-localization of control phage and prlR was observed (data not shown) Taken together, these data indicate that the peptide CRVASVLPC (SEQ ID NO 30) targets prlR in the pancreas On a larger context, these results offer a proof-of-concept biochemical validation for the methodology presented here

TABLE 2
Candidate mouse proteins mimicked by tissue-specific peptides

| Extended Motif | Mouse Protein Containing Motif | Protein Description | Accession # |
|---|---|---|---|
| Muscle | | | |
| DFSGIA+, DFSGIA+ | ADAM 10, Spi12 | Notch Interactor, Serine protease inhibitor (serpin) | NP_031425, NP_035584 |
| GRSG+R, SGASAV | Syndactylism, Jagged 2-similar | Notch ligand | XP_192739 |
| SG+GVF | DPP IV | Dipeptidylpeptidase active in muscle vasculature | NP_034204 |
| AGSF | Fibrillin-1 | Extracellular matrix protein, TGFβ interactor | XP_192917 |
| SLGSFP | SPARC-related protein | Extracellular matrix calcium-binding protein | NP_071711 |
| Pancreas | | | |
| LVSA, WSGL | Placental lactogen-1 α (PL-1) | Pancreas-signaling peptide hormone | AAN39710 1 |
| GWSG | Prolactin-like protein M | Pancreas-signaling peptide hormone | NP_064375 |
| +SVLTR | Ecgf1, ghostatin | Endothelial cell growth factor | NP_612175 1 |
| Brain | | | |
| SLGG | NGF-alpha, kallikrein K22 | Nerve growth factor, NGF endopeptidase | NP_035045, P15948 |
| Kidney | | | |
| GSLS | Endothelin-converting enzyme | Processing of peptidic vasoconstricting hormones | XP_131743 2 |
| LSLSL | Thrombospondin | Anti-angiogenic ECM protein, TGFβ activator | AAA50611 1 |
| Uterus | | | |
| +PGSSF | Pregnancy zone protein, α1M | Differentially-expressed endometrial LRP subunit | NP_031402 1 |
| GSS+WA | Fractalkine, neurotactin | Chemokine, small inducible cytokine | NP_033168 |
| PGLL | Luteinizing hormone β | Pregnancy peptide hormone | NP_032523 |
| Bowel | | | |
| AGVGV | Fibrillin-2 | Extracellular matrix protein, TGFβ interactor | AAA74908 |
| +CFGG+ | Prepronatriodilatin | Atrial natriuretic factor intestinal paracrine effector | P05125 |

For sequence similarity search to mouse proteins, tripeptide-containing motifs (in either orientation) identified in FIG. 3 were screened using BLAST (NCBI) Examples of candidate proteins potentially mimicked by the peptides homing to mouse tissues are listed Sequences correspond to the regions of 100% identity between the peptide selected and the candidate protein Conserved amino acid substitutions are indicated as (+) Tripeptides shown in Table 1 are highlighted

Synchronous phage library screening in vivo It was reasoned that peptide-displaying phage clones of systemically administered library would segregate in the bloodstream irrespective of each other and, thus, target individual organs independently If this hypothesis is correct, independent enrichment of phage-peptides targeting any number of organs should take place upon successive rounds of selection To identify organ-homing motifs, the DNA corresponding to peptide inserts from 96 recovered phage clones were sequenced after each of the three rounds of selection for each of the six organs and the sequences analyzed Preferential cell binding of specifically-homing peptides to differentially-expressed receptors results in enrichment, defined by the increased frequency of the peptide recovery in each subsequent round of the screen (Kolonin et al, 1993, Pasqualini et al, 2001) Thus, a profile the differential distribution of library-encoded peptides among the six organs was studied

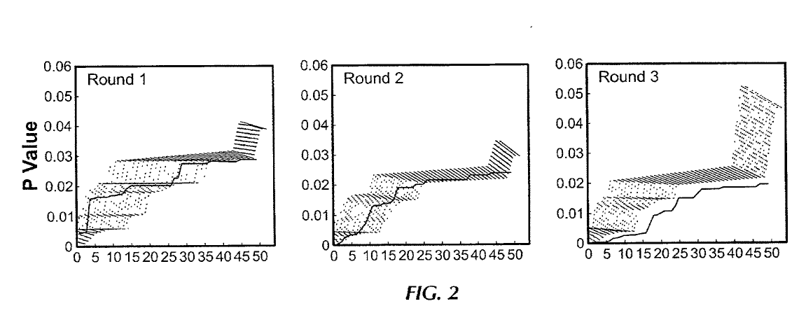

To analyze the spectrum of the peptides resulting from the screening and to compare those among different organs, a combmatonal statistical approach was adopted based on the premise that three residue motifs (tripeptides) provide a sufficient structure for peptide-protein interactions in the context of phage display (Arap et al, 2002) Since a tripeptide within a CX₇C sequence may be responsible for receptor targeting in either orientation, a computer-assisted analysis of the 7,489 tripeptides contained in each direction within the CX₇C inserts (2,620 from the first round, 2,554 from the second round, and 2,315 from the third round) was performed First, the increase in recovery frequency for individual tripeptides in the three consecutive rounds of selection by using the Bayesian Beta/Binomial model (Lee et al, 1996, Carlin and Sargent, 1996) was performed For each organ, a number of tripeptides were found to be progressively enriched (Table 1A), thus, suggesting their superior affinity and/or specificity Next, 2,315 motifs recovered from the third round of selection were surveyed to identify motifs with terminal frequencies higher than those present in the phage library prior to selection The significance of motif representation increase upon selection was assessed by the Fisher exact test (Table 1 B) To show that the P-value of 0 05 for establishment of selected tripeptides was chosen appropriately, a Monte Carlo algorithm (Gelman and Rubin, 1996) was adapted, and confirmed in the third selection round that the P-values of the actual data were smaller than at least 95% of the simulated Pvalues (FIG. 2) Of note, Monte Carlo simulations showed a progressive accumulation of tripeptides isolated with lower P-values from the first to the third round (FIG. 2), consistent with enrichment of the corresponding motifs identified by the Bayesian Beta/Binomial model (Table 1A) Finally, a Fisher exact test was used to analyze the motifs recovered from the third selection round for specificity of tissue homing by identifying tripeptides that were enriched in one of the six organs, but not in the rest of the organs studied (Table 1C)

Identification of candidate biological ligands mimicked by homing peptides The majority of peptide motifs identified by statistical analysis enriched during the screen also showed specificity of association with the organ from which they were recovered (Table 1) For validation of potentially specific organ-homing motifs, the inventors chose to focus on tripeptides that fulfilled the criteria of the statistical tests applied (Table 1) Since peptide motifs binding to cell surface receptors have been previously found to mimic native ligands for these receptors (Kolonin et al, 2002, Arap et al, 2002, Giordano et al, 2001), the ClustalW software was used to determine whether the tripeptides represented parts of longer motifs responsible for organ homing, which would facilitate peptide/protein similarity search For some of the tripeptides, this analysis identified extended (four to seven residue) motifs shared among multiple CX₇C peptides isolated from the given organ (FIG. 3) Each of these extended motifs was screened by using the BLAST algorithm against a non-redundant database of mouse proteins to identify regions of similarity within proteins potentially mimicked by the motifs (Table 2) BLAST output was systematically analyzed for selected motif similarities to extracellular signaling factors that had been reported to regulate organ-dependent vascular growth or homeostasis This revealed 19 motifs as segments of such proteins and, in some cases, identified several motifs that homed to the same organ and that matched different domains within the same protein (Table 2) For example, muscle-homing motifs GRSG+R (SEQ ID NO 13) and SGASAV (SEQ ID NO 14), matched to two different domains of the Jagged2-like protein (Table 2) that belongs to a family of ligands for Notch receptors known to regulate vascular development and function (Linder et al, 2001, Krebs et al, 2000) Similarly, pancreas-homing motifs ASVL (SEQ ID NO 35) (in the reverse orientation) and WSGL (SEQ ID NO 19) showed close similarity to different domains of a placental lactogen (Wiemers et al, 2003) Interestingly, for muscle and pancreas, the inventors also matched homing tripeptides to different ligands that share a receptor with a functional role in vascular biology in the target organ (Table 2) Among skeletal muscle-homing motifs, tripeptides FSG and SGI were partially overlapping in the extended DFSGIA+ (SEQ ID NO 12) region of similarity to disintegrin family metalloproteinases ADAM and Spi 12, respectively, which cleave Notch receptors (Brou et al, 2000) In the pancreas, motif SWSG (SEQ ID NO 32) matched to prolactin (PRL)-like protein M (PLP-M), which belongs to the same family as the placental lactogen PL-I (containing the reverse ASVL (SEQ ID NO 36) and WSGL (SEQ ID NO 19)) and also binds to the PRL receptor (Wiemers et al, 2003, Goffin et al, 2002)

Biochemical validation of PRLR as the target for pancreas-homing peptides To demonstrate the possibility of efficient characterization of circulation-accessible receptors by synchronous biopanning, as a proof-of-principle, the inventors chose to validate the PRL receptor (PRLR) as a peptide target in the pancreas PL-I and PLP-M identified by BLAST analysis belong to the conserved family of PRL-like peptidic hormones that have been shown to function in the pancreas dunng pregnancy (Wiemers et al, 2003, Freemark et al, 2002) Because PRLR is the only known receptor for these proteins (Wiemers et al, 2003, Goffin et al, 2002), It was proposed that the ASVL (SEQ ID NO 36), WSGL (SEQ ID NO 19), and SWSG (SEQ ID NO 32) motifs target PRLR in vivo by mimicking PRL family hormones

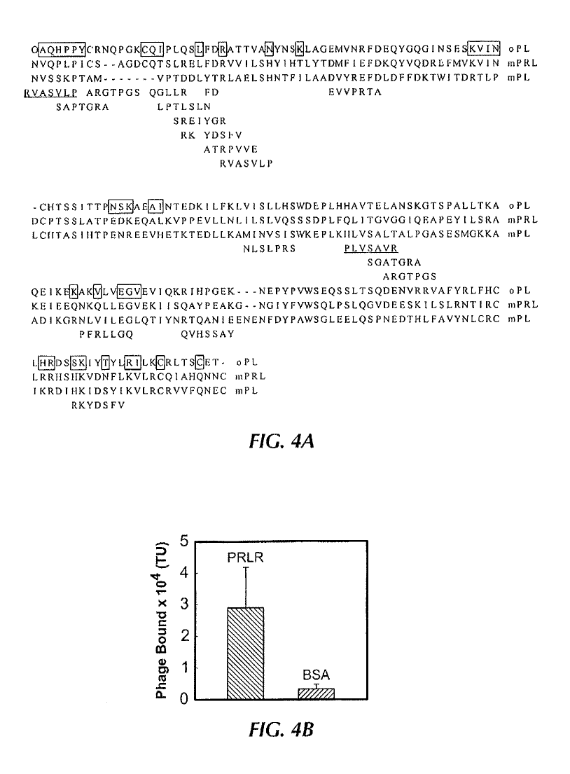

To test this, the inventors attempted to reveal a biochemical interaction of pancreas-homing motifs with PRLR BRASIL (biopanning and rapid analysis of selective interactive ligands) method was used to screen a pancreas-homing phage sub-library (pooled clones recovered in rounds 2 and 3) against PRLR expressed on the surface of COS-1 cells (Wang et al, 1997) In parallel, the same sub-library was screened on purified recombinant PRLR (Bignon et al, 1994) A single round of each selection for PRLR-binding phagepeptides resulted in over 90 percent of the clones sequenced comprised by seven different peptides, five of which were enriched on both immobilized and cell surfaceexpressed PRLR (FIG. 4A) Phage displaying these peptides had specific affinity to PRLR, as determined by subjecting the same sub-library to binding of an immobilized bovine serum albumin (BSA) control (FIG. 4B) Remarkably, computer-assisted analysis of sequences revealed that all of the selected peptides contained amino acid motifs similar to those present in proteins of PRL family (FIG. 4A) Furthermore, there was a clear cluster of matches identified around one of the hormone domains that had been shown (Elkins et al, 2000) to mediate receptor interaction (FIG. 4A) As a negative control, the same similanty search algorithm did not reveal such matches for the selected sequences to unrelated proteins such as insulin, IL-11 and bZIP (data not shown)

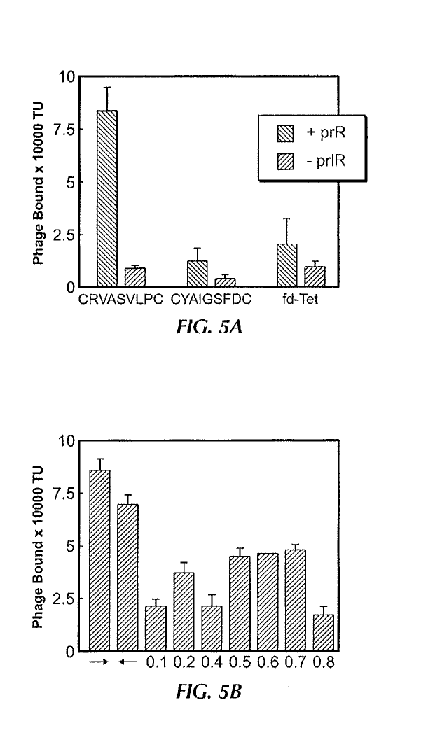

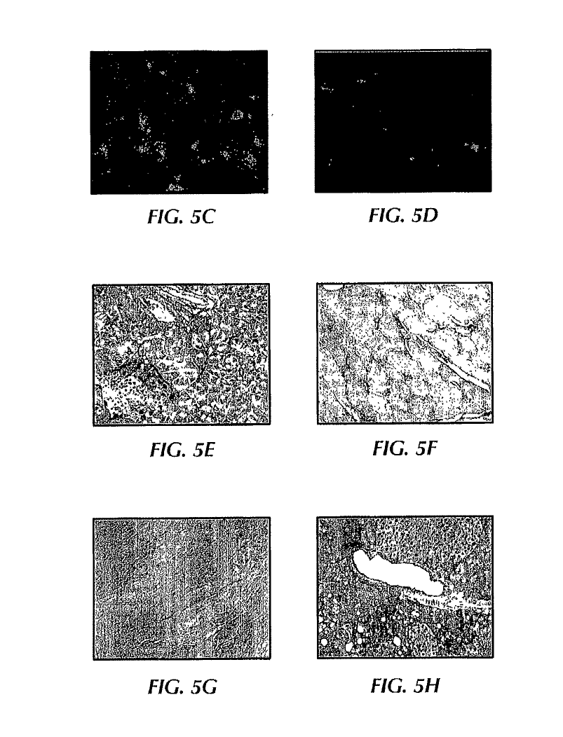

Peptide motif CRVASVLPC (SEQ ID NO 30) recovered as a prolactin binder (FIG. 4) contained a tripeptide, SVL, also identified as one of those enriched in the pancreas during the screen (Table 1) The CRVASVLPC (SEQ ID NO 30) motif matched one of the PL-I sites involved in PRLR interaction (Elkins at al, 2000), as it had amino acids identical to those found in one or more of the three aligned PRL homologues in four out of seven positions (FIG. 4A) Similarity of this peptide in reverse orientation to a part of PL-I (FIG. 4A), initially identified by the BLAST analysis (Table 2), was found by RasMol-assisted analysis of 3D protein structure to reside within the domain exposed on the surface of PRL family proteins (data not shown) To demonstrate direct physical interaction between CRVASVLPC (SEQ ID NO 30) and PRLR, the inventors tested binding of CRVASVLPC(SEQ ID NO 30)-phage to COS-1 cells transfected with PRLR and found it to be 9-fold higher than its nonspecific binding to non-transfected COS-1 cells that served as a negative control (FIG. 5A) To address the issue of a possible importance of the motif orientation for PRLR binding, phage displaying CPLVSAVRC (SEQ ID NO 37) were contructed, the PRL-mimicking motif in the opposite onentation Reversal of the motif did not significantly decrease its binding to PRLR on the expressing cells (FIG. 5B) However, disruption of the motif by alanine-scanning mutagenesis of any amino acid significantly decreased binding to PRLR-expressing cells (FIG. 5A), thus indicating cooperation of the RVASVLP residues compnsing the motif in the receptor recognition To further demonstrate the specific affinity of the CRVASVLPC (SEQ ID NO 30) motif for its receptor, it was shown that the PRL mimic specifically binds to cells expressing PRLR by using immunofluorescence (FIG. 5C) Phage displaying either CRVASVLPC (SEQ ID NO 30) or CPLVSAVRC (SEQ ID NO 37) were found bound and internalized specifically by cells expressing PRLR, but not by non-expressing control cells, whereas none of the CRVASVLPC (SEQ ID NO 30) mutants displayed detectable PRLR-expressing cell binding and internalization (FIGS. 5C-5D and data not shown)

Since the SVL tripeptide found within the PRL mimetopc CRVASVLPC (SEQ ID NO 30) was isolated from the pancreas, the inventors evaluated weather the motif homes to PRLR in the pancreatic blood vessels The inventors showed that the previously reported pancreatic expression of mouse PRLR protein in the vasculature and in the pancreatic islets (Brelje et al, 2002) closely resembles the zn vivo distnbution of phage displaying the CRVASVLPC (SEQ ID NO 30) motif (FIG. 5E-H) Immunohistochemistry of mouse tissues upon intravenous CRVASVLPC (SEQ ID NO 30) phage administration revealed localization of the CRVASVLPC (SEQ ID NO 30) motif to pancreatic blood vessels and the pancreatic islet cells (FIG. 5E), but not to skeletal muscle (FIG. 5F) In contrast, a control in vivo-administered phage clone displaying CYAIGSFDC (SEQ ID NO 31) sequence homing to the skeletal muscle was found predominantly in the vasculature of the skeletal muscle, but not in the pancreas (FIG. 5H) Taken together, these data indicate that the peptide CRVASVLPC (SEQ ID NO 30) binds to PRLR and suggests that it targets vasculature-exposed PRLR in the pancreas

All of the compositions, methods and apparatus disclosed and claimed herein can be made and executed without undue experimentation in light of the present disclosure While the compositions and methods of this invention have been described in terms of preferred embodiments, it will be apparent to those of skill in the art that variations may be applied to the compositions, methods and apparatus and in the steps or in the sequence of steps of the methods described herein without departing from the concept, spirit and scope of the invention More specifically, it are apparent that certain agents that are both chemically and physiologically related may be substituted for the agents described herein while the same or similar results would be achieved All such similar substitutes and modifications apparent to those skilled in the art are deemed to be within the spirit, scope and concept of the invention as defined by the appended claims

### REFERENCES

The following references, to the extent that they provide exemplary procedural or other details supplementary to those set forth herein, are specifically incorporated herein by reference

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,472,509

U.S. Pat. No. 5,021,236

U.S. Pat. No. 5,206,347

U.S. Pat. No. 5,223,409

U.S. Pat. No. 5,401,511

U.S. Pat. No. 5,492,807

U.S. Pat. No. 5,603,872

U.S. Pat. No. 5,622,699

U.S. Pat. No. 5,670,312

U.S. Pat. No. 5,705,610

U.S. Pat. No. 5,840,841

U.S. Pat. No. 5,889,155

U.S. Pat. No. 6,068,829

PCT Appln PCT/US01/28124

PCT Appln PCT/US01/28044

Arap et al, Science, 279 377-380, 1998a

Arap et al, Curr Opin Oncol, 10 560-565, 1998b

Arap et al, Nature Med 8, 121-127, 2002

Baichwal and Sugden, In Gene Transfer, Kucherlapati (Ed), NY, Plenum Press, 117-148, 1986

Bakhshi et al, Cell, 41(3) 899-906, 1985

Barany and Merrifield, In The Peptides, Gross and Meienhofer (Eds), Academic Press, NY, 1-284, 1979

Brelje et al, J Histochem Cytochem, 50 365-383, 2002

Brooks et al, Cell, 79 1157-1164, 1994

Brou et al, Mol Cell, 5 207-216, 2000

Burg et al, Cancer Res, 58 2869-2874, 1999

Carlin and Louis, In Bayes and Empirical Bayes Methods for Data Analysis, 2^{nd} Ed, Chapman & Hall/CRC, London, UK, 2000

Chen and Okayama, Mol Cell Biol, 7 2745-2752, 1987

Cleary and Sklar, Proc Natl Acad Set USA, (21) 7439-7443, 1985

Cleary et al, J Exp Med, 164(1) 315-320, 1986

Coffin, In Virology, Fields et al (Eds), Raven Press, NY, 1437-1500, 1990

Coupar et al, Gene, 68 1-10, 1988

Elicein and Cheresh, Curr Opin Cell Biol, 13(5) 563-568, 2001

Ellerby et al, Nature Med, 9 1032-1038, 1999

Folkman, Nature Biotechnol, 15 510, 1997

Folkman, Nature Med, 1 27-31, 1995

Freemark et al, Endocrinology, 143 1378-85, 2002

Friedmann, Science, 244 1275-1281, 1989

Gomez Foix et al, J Biol Chem, 267 25129 25134, 1992

Goodman and Gilman's The Pharmacological Basis Of Therapeutics, Hardman et al (Eds), 10^{th} Ed, 32 853-860, 35 891-893, 2001

Gopal, Mol Cell Biol, 5 1188-1190, 1985

Graham and Prevec, In Methods in Molecular Biology Gene Transfer and Expression Protocol, Murray (Ed), Humana Press, Clifton, N.J., 7 109-128, 1991

Graham and van der Eb, Virology, 52 456-467, 1973

Graham et al, J Gen Virol, 36 59-72, 1977

Grunhaus and Horwitz, Seminar in Virology, 3 237-252, 1992

Harlow and Lane, Antibodies A Laboratory Manual, Cold Spring Harbor Laboratory Press, NY, 1988

Hermonat and Muzycska, Proc Natl Acad Sci USA, 81 6466-6470, 1984

Herz and Gerard, Proc Natl Acad Sci USA, 90 2812 2816, 1993

Horwich, et al, J Virol, 64 642-650, 1990

Johnson et al, In Peptide Turn Mimetics, Pezzuto et al (Eds), Chapman and Hall, NY, 1993

Jones and Shenk, Cell, 13 181 188, 1978

Kerr et al, Br J Cancer, 26(4) 239-257, 1972

Koivunen et al, Methods Mol Biol, 129 3-17, 1999b

Koivunen et al, Nature Biotechnol, 17 768-774, 1999a

Kolonin et al, Curr Opin Chem Biol, 5 308-313, 2001

Kolonin et al, Nature Med, 6 625-632, 2004

Kolonin et al, Proc Natl Acad Sci USA, 99 13055-13060, 2002

Le Gal La Salle et al, Science, 259 988-990, 1993

Levrero et al, Gene, 101 195-202, 1991

Lindner et al, Am J Pathol, 159 875-883, 2001

Manly, In Randomization, bootstrap and Monte Carlo methods in biology, 2nd Ed, Chapman & Hall/CRC, London, UK, 1997

Mann et al, Cell, 33 153-159, 1983

Merrifield, Science, 232 341-347, 1986

Nicolas and Rubinstein, In Vectors A survey of molecular cloning vectors and their uses, Rodriguez and Denhardt (Eds), Stoneham Butterworth, 494-513, 1988

Nicolau et al, Methods Enzymol, 149 157-176, 1987

Paskind et al, Virology, 67 242-248, 1975

Pasqualini and Ruoslahti, Nature, 380 364-366, 1996

Pasqualini et al, Cancer Res, 60 722-727, 2000

Pasqualini et al, In Phage Display A Laboratory Manual Barbas et al, (Eds), 221 22-24, Cold Spring Harbor Laboratory Press, NY, 2001

Pasqualini, J Nucl Med, 43 159-162, 1999

Physicians Desk Reference

Potter et al, Proc Nat Acad Sci USA, 81 7161-7165, 1984

Racher et al, Biotechnology Techniques, 9 169-174, 1995

Ragot et al, Nature, 361 647-650, 1993

Rajotte and Ruoslahti, J Biol Chem, 274 11593-11598, 1999

Rajotte et al, J Clin Invest, 102 430-437, 1998

Remington's Pharmaceutical Sciences, 15^{th} ed, 33 624-652, Mack Publishing Company, Easton, Pa., 1980

Rich et al, Hum Gene Ther, 4 461-476, 1993

Ridgeway, In Vectors A Survey of Molecular Cloning Vectors and Their Uses, Rodriguez et al (Eds), Stoneham Butterworth, 467-492, 1988

Rippe et al, Mol Cell Biol, 10 689-695, 1990

Rosenfeld et al, Cell, 68 143-155, 1992

Rosenfeld et al, Science, 252 431-434, 1991

Smith and Scott, Meth Enzymol, 21 228-257, 1993

Smith and Scott, Science, 228 1315-1317, 1985

Stewart and Young, In Solid Phase Peptide Synthesis, 2^{nd} Ed, Pierce Chemical Co, 1984

Stratford-Perricaudet and Perricaudet, In Human Gene Transfer, Cohen-Haguenauer et al (Eds), John Libbey Eurotext, France, 51-61, 1991

Stratford-Perricaudet et al, Hum Gene Ther, 1 241-256, 1990

Tam et al, J Am Chem Soc, 105 6442, 1983

Temin, In Gene Transfer, Kucherlapati (Ed), NY, Plenum Press, 149-188, 1986

Tsujimoto and Croce, Proc Natl Acad Sci USA, 83(14) 5214-5218, 1986

Tsujimoto et al, Science, 228(4706) 1440-1443, 1985

Tur-Kaspa et al, Mol Cell Biol, 6 716-718, 1986

Wiemers et al, Endocrinology 144 313-325, 2003

Wong et al, Gene, 10 87-94, 1980

Wu and Wu, Biochemistry, 27 887-892, 1988

Wu and Wu, J Biol Chem, 262 4429-4432, 1987

Zurita et al, Cancer Res, 64 435-439, 2004

1. A method of providing peptides that bind to distinct tissues comprising the steps of:
a) administering a phage display library displaying random heterologous peptides to a first subject;
b) obtaining samples of two or more tissues from the first subject;
c) obtaining phage bound to the samples from the first subject;
d) administering the phage obtained in step (c) to a second subject;
e) obtaining samples of two or more selected tissues from the second subject;
f) obtaining phage bound to said samples; and
g) providing peptides having amino acid sequences present on one or more of the bound phage.

2. The method of claim 1, wherein phage obtained from tissues of the second subject in step f) are administered to a third subject, and phage bound to tissues of said third subject are obtained, prior to step g).

3. The method of claim 1, wherein administration of phage is by injection.

4. (canceled)

5. The method of claim 1, wherein the subject is a mammal.

6. The method of claim 5, wherein the mammal is a human.

7-8. (canceled)

9. The method of claim 8, wherein the tissue is muscle, pancreas, brain, kidney, uterus, bowel, intestine, small intestine, heart, artery, vein, aorta, coronary artery, lung, spleen, bone marrow, bladder, prostate, adipose, or ovary.

10. The method of claim 1, further comprising, prior to step g):
i) obtaining a sample of one or more tissues;
ii) contacting the phage obtained from the first or second subject with the sample obtained in i);
iii) obtaining phage that do not bind to said sample.

11. The method of claim 1, further comprising operatively coupling the peptide to an agent to be delivered to tissues of a subject and administering the peptide-coupled agent to the subject.

12-13. (canceled)

14. The method of claim 11, wherein the agent is a drug, a chemotherapeutic agent, a radioisotope, a pro-apoptosis agent, an anti-angiogenic agent, an enzyme, a hormone, a cytokine, a growth factor, a cytotoxic agent, a peptide, a protein, an antibiotic, an antibody, a Fab fragment of an antibody, an imaging agent, an antigen, a survival factor, an anti-apoptotic agent, a hormone antagonist, a virus, a bacteriophage, a bacterium, a liposome, a microparticle, a magnetic bead, a microdevice, a yeast cell, a mammalian cell, a cell or an expression vector.

15. An isolated tissue-targeting peptide of 100 amino acids or less in size, comprising at least 3 contiguous amino acids of a sequence is selected from:
a) a first group of muscle-targeting peptide sequences consisting of Ala-Pro-Ala (APA), Arg-Ser-Gly (RSG), Ser-Gly-Ala (SGA), Ala-Ile-Gly (AIG), Ile-Gly-Ser (IGS), Gly-Ser-Phe (GSF), Ala-Gly-Gly (AGG), Ala-Ser-Arg (ASR), Asp-Phe-Ser (DFS), Asp-Gly-Thr (DGT), Asp-Thr-Gly (DTG), Phe-Arg-Ser (FRS), Gly-Asp-Thr (GDT), Gly-Gly-Thr (GGT), Gly-Trp-Ser (GWS), Ile-Ala-Tyr (IAY), Arg-Arg-Ser (RRS), and Ser-Gly-Val (SGV);
b) a second group of pancreas-targeting peptide sequences consisting of Leu-Val-Ser (LVS), Val-Ser-Ser (VSS), Trp-Ser-Gly (WSG), Gly-Trp-Arg (GWR), Gly-Tyr-Asn (GYN), Leu-Thr-Arg (LTR), Thr-Leu-Val (TLV), and Phe-Gly-Val (FGV);
c) a third group of brain-targeting peptide sequences consisting of Leu-Gly-Gly (LGG), Arg-Gly-Phe (RGF), Ala-Leu-Gly (ALG), Leu-Leu-Ser (LLS), Asp-Ser-Tyr (DSY), Gly-Phe-Ser (GFS), Gly-Ile-Trp (GIW), and His-Gly-Leu (HGL);
d) a fourth group of kidney-targeting peptide sequences consisting of Leu-Gly-Ser (LGS), Ser-Leu-Ser (SLS), Asp-Arg-Gly (DRG), Arg-Arg-Val (RRV), Asp-Ser-Gly (DSG), Leu-Arg-Val (LRV), Ser-Arg-Val (SRV), and Phe-Leu-Ser (FLS);
e) a fifth group of uterus-targeting peptide sequences consisting of Gly-Ser-Ser (GSS), Leu-Leu-Gly (LLG), Gly-Ala-Ala (GAA), Gly-Leu-Leu (GLL), Ala-Arg-Gly (ARG), Gly-Ala-Ser (GAS), Gly-Gly-Leu (GGL), and Gly-Pro-Ser (GPS);
f) a sixth group of bowel-targeting peptide sequences consisting of Ala-Gly-Val (AGV), Trp-Arg-Asp (WRD), Phe-Gly-Gly (FGG), Gly-Gly-Arg (GGR), Gly-Arg-Val (GRV), Arg-Trp-Ser (RWS), Val-Gly-Val (VGV), and Gly-Val-Gly (GVG);
wherein the tissue-targeting peptide is coupled to a solid support or an agent to be delivered to a tissue, organ or vasculature thereof.

16-21. (canceled)

22. The isolated peptide of claim 15, wherein the peptide is 50 amino acids or less in size.

23. The isolated peptide of claim 22, wherein the peptide is 25 amino acids or less in size.

24. The isolated peptide of claim 23, wherein the peptide is 10 amino acids or less in size.

25. The isolated peptide of claim 24, wherein the peptide is 9 amino acids or less in size.

26. The isolated peptide of claim 25, wherein the peptide is 7 amino acids or less in size.

27. The isolated peptide of claim 26, wherein the peptide is 5 amino acids in size.

28. The isolated peptide of claim 15, wherein the peptide comprises an amino acid sequence is:
a) a muscle-targeting sequence selected from the group consisting of Asp-Phe-Ser-Gly-Ile-Ala-Xaa (SEQ ID NO: 12), Gly-Arg-Ser-Gly-Xaa-Arg (SEQ ID NO: 13), Ser-Gly-Ala-Ser-Ala-Val (SEQ ID NO: 14), Ser-Gly-Xaa-Gly-Val-Phe (SEQ ID NO: 15), Ala-Gly-Ser-Phe (SEQ ID NO: 16), and Ser-Leu-Gly-Ser-Phe-Pro (SEQ ID NO: 17);
b) a pancreas-targeting sequence selected from the group consisting of Leu-Val-Ser-Ala (SEQ ID NO: 18), Trp-Ser-Gly-Leu (SEQ ID NO: 19), Gly-Trp-Ser-Gly (SEQ ID NO: 20), and Xaa-Ser-Val-Leu-Thr-Arg (SEQ ID NO: 21);
c) a brain-targeting sequence of Ser-Leu-Gly-Gly (SEQ ID NO: 22);
d) a kidney-targeting sequence selected from the group consisting of Gly-Ser-Leu-Ser (SEQ ID NO: 23) and Leu-Ser-Leu-Ser-Leu (SEQ ID NO: 24);
e) a uterus-targeting sequence selected from the group consisting of Xaa-Pro-Gly-Ser-Ser-Phe (SEQ ID NO: 25), Gly-Ser-Ser-Xaa-Trp-Ala (SEQ ID NO: 26), Pro-Gly-Leu-Leu (SEQ ID NO: 27); and
f) a bowel-targeting sequence selected from the group consisting of Ala-Gly-Val-Gly-Val (SEQ ID NO: 28), and Xaa-Cys-Phe-Gly-Gly-Xaa (SEQ ID NO: 29);
wherein Xaa is a positively charged amino acid.

29-34. (canceled)

35. The isolated peptide of claim 15, wherein the peptide is covalently coupled to the agent to be delivered.

36. The isolated peptide of claim 35, wherein the agent is a drug, a chemotherapeutic agent, a radioisotope, a pro-apoptotic agent, an anti-angiogenic agent, a hormone, a cytokine, a growth factor, a cytotoxic agent, a peptide, a protein, an antibiotic, an antibody, a Fab fragment of an antibody, an imaging agent, survival factor, an anti-apoptotic agent, a hormone antagonist or an antigen.

37. The isolated peptide of claim 36, wherein the pro-apoptotic agent is selected from the group consisting of gramicidin, magainin, mellitin, defensin, cecropin, (KLAKLAK)₂ (SEQ ID NO:1), (KLAKKLA)₂ (SEQ ID NO:2), (KAAKKAA)₂ (SEQ ID NO:3) and (KLGKKLG)₃ (SEQ ID NO:4).

38. The isolated peptide of claim 36, wherein the anti-angiogenic agent is selected from the group consisting of thrombospondin, angiostatin 5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4 and minocycline.

39. The isolated peptide of claim 36, wherein the cytokine is selected from the group consisting of interleukin 1 (IL-1), IL-2, IL-5, IL-10, IL-11, IL-12, IL-18, interferon-γ (IF-γ), IF-α, IF-β, tumor necrosis factor-α (TNF-α), or GM-CSF (granulocyte macrophage colony stimulating factor).

40. The isolated peptide of claim 35, wherein the agent is a virus, a bacteriophage, a bacterium, a liposome, a microparticle, a magnetic bead, a yeast cell, a mammalian cell or a cell.

41. The isolated peptide of claim 40, wherein the virus is a lentivirus, a papovaviruses, a simian virus 40, a bovine papilloma virus, a polyoma virus, adenovirus, vaccinia virus, adeno-associated virus (AAV), or herpes virus.

42. The isolated peptide of claim 40, wherein the agent is a eukaryotic expression vector.

43. The isolated peptide of claim 42, wherein the vector is a gene therapy vector.

44. The isolated peptide of claim 15, wherein the peptide is attached to a solid support.

45. A method of delivering an agent to a tissue comprising obtaining a peptide coupled to such an agent in accordance with claim 15 and contacting the tissue with said peptide-coupled agent.

46. The method of claim 45, wherein the tissue is located in a human patient.

47. (canceled)
